SEQUENCE LISTING (88 sequences)

SEQ ID NO: 1 - protein, 5 aa, synthetic construct
SYGMH

SEQ ID NO: 2 - protein, 17 aa, synthetic construct
IIWFDGSSTYYADSVRG

SEQ ID NO: 3 - protein, 9 aa, synthetic construct
ELGRRYFDL

SEQ ID NO: 4 - protein, 11 aa, synthetic construct
RASQSVSSYLA

SEQ ID NO: 5 - protein, 7 aa, synthetic construct
DASKRAT

SEQ ID NO: 6 - protein, 11 aa, synthetic construct
QQRSKWPPWTF

SEQ ID NO: 7 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 8 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 9 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
GYTFTSYW

SEQ ID NO: 12 - protein, 8 aa, synthetic construct
INPSNGRT

SEQ ID NO: 13 - protein, 17 aa, synthetic construct
ARGRPDYYGSSKWYFDV

SEQ ID NO: 14 - protein, 11 aa, synthetic construct
QSIVHSNVNTY

SEQ ID NO: 15 - skipped

SEQ ID NO: 16 - protein, 9 aa, synthetic construct
FQGSHVPPT

SEQ ID NO: 17 - protein, 124 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 18 - protein, 113 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 19-20 - skipped (2 entries)

SEQ ID NO: 21 - protein, 9 aa, synthetic construct
GYSITGGYL

SEQ ID NO: 22 - protein, 7 aa, synthetic construct
ISYDGTN

SEQ ID NO: 23 - protein, 10 aa, synthetic construct
ARYGRVFFDY

SEQ ID NO: 24 - protein, 11 aa, synthetic construct
QSIVHSNGNTY

SEQ ID NO: 25 - skipped

SEQ ID NO: 26 - protein, 9 aa, synthetic construct
FQGSHVPWT

SEQ ID NO: 27 - protein, 117 aa, synthetic construct [sequence omitted]

SEQ ID NO: 28 - protein, 113 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKR

SEQ ID NO: 29-30 - skipped (2 entries)

SEQ ID NO: 31 - protein, 8 aa, synthetic construct
GGTFSSYA

SEQ ID NO: 32 - protein, 8 aa, synthetic construct
IIPIFGTA

SEQ ID NO: 33 - protein, 23 aa, synthetic construct
ARAPLRFLEWSTQDHYYYYYMDV

SEQ ID NO: 34 - protein, 6 aa, synthetic construct
SLRSYY

SEQ ID NO: 35 - skipped

SEQ ID NO: 36 - protein, 11 aa, synthetic construct
KSRDGSGQHLV

SEQ ID NO: 37 - protein, 130 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 38 - protein, 112 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPK

SEQ ID NO: 39-40 - skipped (2 entries)

SEQ ID NO: 41 - protein, 8 aa, synthetic construct
GFTFSSYA

SEQ ID NO: 42 - protein, 8 aa, synthetic construct
ISGSGGTT

SEQ ID NO: 43 - protein, 18 aa, synthetic construct
AKDLGWSDSYYYYYGMDV

SEQ ID NO: 44 - protein, 6 aa, synthetic construct
QGIRND

SEQ ID NO: 45 - skipped

SEQ ID NO: 46 - protein, 9 aa, synthetic construct
LQHNSYPCS

SEQ ID NO: 47 - protein, 125 aa, synthetic construct [sequence omitted]

SEQ ID NO: 48 - protein, 107 aa, synthetic construct [sequence omitted]

SEQ ID NO: 49-50 - skipped (2 entries)

SEQ ID NO: 51 - protein, 9 aa, synthetic construct
GGSISSSNW

SEQ ID NO: 52 - protein, 7 aa, synthetic construct
IYHSGST

SEQ ID NO: 53 - protein, 12 aa, synthetic construct
ARWTGRTDAFDI

SEQ ID NO: 54 - protein, 11 aa, synthetic construct
QSLLHSNGYNY

SEQ ID NO: 55 - skipped

SEQ ID NO: 56 - protein, 9 aa, synthetic construct
MQGTHWPLT

SEQ ID NO: 57 - protein, 119 aa, synthetic construct [sequence omitted]

SEQ ID NO: 58 - protein, 112 aa, synthetic construct [sequence omitted]

SEQ ID NO: 59-60 - skipped (2 entries)

SEQ ID NO: 61 - protein, 8 aa, synthetic construct
GFTFSSFA

SEQ ID NO: 62 - protein, 7 aa, synthetic construct
IDTRGAT

SEQ ID NO: 63 - protein, 12 aa, synthetic construct
ARLGNFYYGMDV

SEQ ID NO: 64 - protein, 6 aa, synthetic construct
QSIGSS

SEQ ID NO: 65 - skipped

SEQ ID NO: 66 - protein, 9 aa, synthetic construct
HQSSRLPHT

SEQ ID NO: 67 - protein, 118 aa, synthetic construct [sequence omitted]

SEQ ID NO: 68 - protein, 107 aa, synthetic construct [sequence omitted]

SEQ ID NO: 69-70 - skipped (2 entries)

SEQ ID NO: 71 - protein, 8 aa, synthetic construct
GFTFSIYR

SEQ ID NO: 72 - protein, 8 aa, synthetic construct
ISPSGGTT

SEQ ID NO: 73 - protein, 13 aa, synthetic construct
ARWSGGSGYAFDI

SEQ ID NO: 74 - protein, 6 aa, synthetic construct
RDIRNY

SEQ ID NO: 75 - skipped

SEQ ID NO: 76 - protein, 9 aa, synthetic construct
QQFDSLPHT

SEQ ID NO: 77 - protein, 120 aa, synthetic construct
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 78 - protein, 107 aa, synthetic construct
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 79-80 - skipped (2 entries)

SEQ ID NO: 81 - protein, 8 aa, synthetic construct
GFTFSSYA

SEQ ID NO: 82 - protein, 8 aa, synthetic construct
ISGSGGTT

SEQ ID NO: 83 - protein, 18 aa, synthetic construct
AKDLGWSDSYYYYYGMDV

SEQ ID NO: 84 - protein, 6 aa, synthetic construct
QGIRND

SEQ ID NO: 85 - skipped

SEQ ID NO: 86 - protein, 9 aa, synthetic construct
LQHNSYPSS

SEQ ID NO: 87 - protein, 125 aa, synthetic construct [sequence omitted]

SEQ ID NO: 88 - protein, 107 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPSSFGQGTKLEIK

(19) United States
(12) Patent Application Publication
(10) Pub. No.: US 2024/0109970 A1
(43) Pub. Date: Apr. 4, 2024

(54) METHODS FOR TREATING INACTIVE THYROID EYE DISEASE

(71) Applicant: Horizon Therapeutics Ireland DAC, Dublin 2 (IE)

(72) Inventors: Elizabeth H.Z. THOMPSON, Pacifica, CA (US); Jeffrey Wayne SHERMAN, Lincolnshire, IL (US)

(21) Appl. No.: 18/477,140

(22) Filed: Sep. 28, 2023

Related U.S. Application Data
(60) Provisional application No. 63/495,033, filed on Apr. 7, 2023.
(60) Provisional application No. 63/377,870, filed on Sep. 30, 2022.

(51) Int. Cl. C07K 16/28; A61P 27/02

(52) U.S. Cl. CPC C07K 16/2863; A61P 27/02; A61K 2039/545; C07K 2317/76

(57) ABSTRACT

Described herein is a method of treating an individual with inactive thyroid eye disease (TED), the method comprising administering to an individual with inactive or chronic TED an insulin-like growth factor 1 receptor (IGF1R) inhibitor, thereby treating the inactive or chronic TED.

### CROSS-REFERENCE

This application claims the benefit of U.S. Provisional Application No. 63/495,033, filed Apr. 7, 2023, and U.S. Provisional Application No. 63/377,870, filed Sep. 30, 2022, the contents of which are incorporated by reference in their entirety.

### SEQUENCE LISTING

The instant application contains a Sequence Listing which has been submitted electronically in XML and is hereby incorporated by reference in its entirety. Said XML copy, created on Sep. 28, 2023, is named 58651-826_201_SL.xml and is 65,536 bytes in size.

### BACKGROUND

Thyroid Eye Disease (TED) is a disorder that can lead to bulging eyes (proptosis), double vision (diplopia), and ultimately blindness if left inadequately treated or untreated. TED can be divided into an active phase (also referred to as an acute phase) and an inactive phase (also referred to as a chronic phase). The active phase can last anywhere from approximately 6 months to 2 years. The active phase is followed by the inactive phase in which disease progression halts. However, symptoms such as diplopia and proptosis can remain after disease progression has plateaued. A major etiological mechanism of TED development has been attributed to signaling through the insulin-like growth factor 1 receptor (IGF1R). Inhibition of IGF1R signaling has been shown to reduce both diplopia and proptosis during the active phase of disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Described herein are methods of treating inactive and/or chronic forms of Thyroid Eye Disease (TED) using insulin-like growth factor 1 receptor (IGF1R) inhibitors. Such methods provide the benefit of potentially prolonging an inactive state of the disease and/or preventing development of an acute/active phase from an inactive phase. It is currently unclear whether patients with low clinical activity scores (CAS; e.g., CAS less than or equal to 1) or patients who have experienced chronic disease (e.g., diagnosis of greater than 1 year) would benefit from treatment with IGF1R inhibitors such as teprotumumab. In certain cases, the methods describe herein can treat patients with low CAS, patients with chronic disease, or patients with low CAS and chronic disease. Importantly, the methods described herein can increase the quality of life of TED patients as measured by the GO-QoL assessment.

Described herein in one aspect is a method of treating an individual with inactive thyroid eye disease, the method comprising administering to an individual with inactive or chronic thyroid eye disease (TED) an IGF1R inhibitor, thereby treating the inactive or chronic TED. In certain embodiments, the inactive TED is associated with Grave's disease. In certain embodiments, the inactive TED has a Clinical Activity Score (CAS) of 1 or less. In certain embodiments, the inactive or chronic TED has a Clinical Activity Score (CAS) of 0. In certain embodiments, the inactive TED is chronic TED. In certain embodiments, the chronic TED has been inactive for at least 6 months prior to treatment. In certain embodiments, the inactive or chronic TED has been inactive or chronic for at least 12 months prior to treatment. In certain embodiments, the inactive or chronic TED has been previously treated with an IGF1R inhibitor. In some cases, the inactive or chronic TED has not been previously treated with an IGF1R inhibitor. In some cases, the individual with the inactive or chronic TED has not previously undergone orbital irradiation. In some cases, the individual with the inactive or chronic TED has not previously undergone orbital decompression surgery. In some cases, the individual with the inactive or chronic TED has not previously undergone strabismus surgery. In certain embodiments, the IGF1R inhibitor comprises ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof. In certain embodiments, the IGF1R inhibitor comprises an antibody that binds IGF1R. In certain embodiments, the antibody that binds IGF1R is chimeric or humanized. In certain embodiments, the antibody that binds IGF1R is an IgG antibody. In certain embodiments, the antibody that binds IGF1R is a Fab, F(ab)₂, a single-domain antibody, or a single chain variable fragment (scFv). In certain embodiments, the IGF1R inhibitor is an antibody comprising ganitumab, figitumumab, MEDI-573, cixutumumab, dalotuzumab, robatumumab, robatumumab, AVE1642, BIIB022, xentuzumab, istiratumab, or any combination thereof. In certain embodiments, the antibody that binds IGF1R comprises: (a) an immunoglobulin heavy chain CDR1 (CDR-H1) comprising the amino acid sequence set forth in any one of SEQ ID NO: 1, 11, 21, 31, 41, 51, 61, 71, or 81; (b) an immunoglobulin heavy chain CDR2 (CDR-H2) comprising the amino acid sequence set forth in any one of SEQ ID NO: 2, 12, 22, 32, 42, 52, 62, 72, or 82; (c) an immunoglobulin heavy chain CDR3 (CDR-H3) comprising the amino acid sequence set forth in any one of SEQ ID NO: 3, 13, 23, 33, 43, 53, 63, 73, or 83; (d) an immunoglobulin light chain CDR1 (CDR-L1) comprising the amino acid sequence set forth in any one of SEQ ID NO: 4, 14, 24, 34, 44, 54, 64, 74, or 84; (e) an immunoglobulin light chain CDR2 (CDR-L2) amino comprising the amino acid sequence set forth in any one of SEQ ID NO: 5, 15, 25, 35, 45, 55, 65, 75, or 85; and/or (f) and an immunoglobulin light chain CDR3 (CDR-L3) comprising the amino acid sequence set forth in any one of SEQ ID NO: 6, 16, 26, 36, 46, 56, 66, 76, or 86. In certain embodiments, the antibody that binds IGF1R comprises: (a) an immunoglobulin heavy chain CDR1 (CDR-H1) comprising the amino acid sequence set forth in SEQ ID NO: 1; (b) an immunoglobulin heavy chain CDR2 (CDR-H2) comprising the amino acid sequence set forth in SEQ ID NO: 2; (c) an immunoglobulin heavy chain CDR3 (CDR-H3) comprising the amino acid sequence set forth in SEQ ID NO: 3; (d) an immunoglobulin light chain CDR1 (CDR-L1) comprising the amino acid sequence set forth in SEQ ID NO: 4; (e) an immunoglobulin light chain CDR2 (CDR-L2) amino comprising the amino acid sequence set forth in SEQ ID NO: 5; and/or (f) and an immunoglobulin light chain CDR3 (CDR-L3) comprising the amino acid sequence set forth in SEQ ID NO: 6. In certain embodiments, the antibody that binds IGF1R comprises: (a) an immunoglobulin heavy chain CDR1 (CDR-H1) comprising the amino acid sequence set forth in SEQ ID NO: 11; (b) an immunoglobulin heavy chain CDR2 (CDR-H2) comprising the amino acid sequence set forth in SEQ ID NO: 12; (c) an immunoglobulin heavy chain CDR3 (CDR-H3) comprising the amino acid sequence set forth in SEQ ID NO: 13; (d) an immunoglobulin light chain CDR1 (CDR-L1) comprising the amino acid sequence set forth in SEQ ID NO: 14; (e) an immunoglobulin light chain CDR2 (CDR-L2) amino comprising the amino acid sequence set forth in SEQ ID NO: 15; and/or (f) and an immunoglobulin light chain CDR3 (CDR-L3) comprising the amino acid sequence set forth in SEQ ID NO: 16. In certain embodiments, the antibody that binds IGF1R comprises: (a) an immunoglobulin heavy chain CDR1 (CDR-H1) comprising the amino acid sequence set forth in SEQ ID NO: 21; (b) an immunoglobulin heavy chain CDR2 (CDR-H2) comprising the amino acid sequence set forth in SEQ ID NO: 22; (c) an immunoglobulin heavy chain CDR3 (CDR-H3) comprising the amino acid sequence set forth in SEQ ID NO: 23; (d) an immunoglobulin light chain CDR1 (CDR-L1) comprising the amino acid sequence set forth in SEQ ID NO: 24; (e) an immunoglobulin light chain CDR2 (CDR-L2) amino comprising the amino acid sequence set forth in SEQ ID NO: 25; and/or (f) and an immunoglobulin light chain CDR3 (CDR-L3) comprising the amino acid sequence set forth in SEQ ID NO: 26. In certain embodiments, the antibody that binds IGF1R comprises: (a) an immunoglobulin heavy chain CDR1 (CDR-H1) comprising the amino acid sequence set forth in SEQ ID NO: 31; (b) an immunoglobulin heavy chain CDR2 (CDR-H2) comprising the amino acid sequence set forth in SEQ ID NO: 32; (c) an immunoglobulin heavy chain CDR3 (CDR-H3) comprising the amino acid sequence set forth in SEQ ID NO: 33; (d) an immunoglobulin light chain CDR1 (CDR-L1) comprising the amino acid sequence set forth in SEQ ID NO: 34; (e) an immunoglobulin light chain CDR2 (CDR-L2) amino comprising the amino acid sequence set forth in SEQ ID NO: 35; and/or (f) and an immunoglobulin light chain CDR3 (CDR-L3) comprising the amino acid sequence set forth in SEQ ID NO: 36. In certain embodiments, the antibody that binds IGF1R comprises: (a) an immunoglobulin heavy chain CDR1 (CDR-H1) comprising the amino acid sequence set forth in SEQ ID NO: 41; (b) an immunoglobulin heavy chain CDR2 (CDR-H2) comprising the amino acid sequence set forth in SEQ ID NO: 42; (c) an immunoglobulin heavy chain CDR3 (CDR-H3) comprising the amino acid sequence set forth in SEQ ID NO: 43; (d) an immunoglobulin light chain CDR1 (CDR-L1) comprising the amino acid sequence set forth in SEQ ID NO: 44; (e) an immunoglobulin light chain CDR2 (CDR-L2) amino comprising the amino acid sequence set forth in SEQ ID NO: 45; and/or (f) and an immunoglobulin light chain CDR3 (CDR-L3) comprising the amino acid sequence set forth in SEQ ID NO: 46. In certain embodiments, the antibody that binds IGF1R comprises: (a) an immunoglobulin heavy chain CDR1 (CDR-H1) comprising the amino acid sequence set forth in SEQ ID NO: 51; (b) an immunoglobulin heavy chain CDR2 (CDR-H2) comprising the amino acid sequence set forth in SEQ ID NO: 52; (c) an immunoglobulin heavy chain CDR3 (CDR-H3) comprising the amino acid sequence set forth in SEQ ID NO: 53; (d) an immunoglobulin light chain CDR1 (CDR-L1) comprising the amino acid sequence set forth in SEQ ID NO: 54; (e) an immunoglobulin light chain CDR2 (CDR-L2) amino comprising the amino acid sequence set forth in SEQ ID NO: 55; and/or (f) and an immunoglobulin light chain CDR3 (CDR-L3) comprising the amino acid sequence set forth in SEQ ID NO: 56. In certain embodiments, the antibody that binds IGF1R comprises: (a) an immunoglobulin heavy chain CDR1 (CDR-H1) comprising the amino acid sequence set forth in SEQ ID NO: 61; (b) an immunoglobulin heavy chain CDR2 (CDR-H2) comprising the amino acid sequence set forth in SEQ ID NO: 62; (c) an immunoglobulin heavy chain CDR3 (CDR-H3) comprising the amino acid sequence set forth in SEQ ID NO: 63; (d) an immunoglobulin light chain CDR1 (CDR-L1) comprising the amino acid sequence set forth in SEQ ID NO: 64; (e) an immunoglobulin light chain CDR2 (CDR-L2) amino comprising the amino acid sequence set forth in SEQ ID NO: 65; and/or (f) and an immunoglobulin light chain CDR3 (CDR-L3) comprising the amino acid sequence set forth in SEQ ID NO: 66. In certain embodiments, the antibody that binds IGF1R comprises: (a) an immunoglobulin heavy chain CDR1 (CDR-H1) comprising the amino acid sequence set forth in SEQ ID NO: 71; (b) an immunoglobulin heavy chain CDR2 (CDR-H2) comprising the amino acid sequence set forth in SEQ ID NO: 72; (c) an immunoglobulin heavy chain CDR3 (CDR-H3) comprising the amino acid sequence set forth in SEQ ID NO: 73; (d) an immunoglobulin light chain CDR1 (CDR-L1) comprising the amino acid sequence set forth in SEQ ID NO: 74; (e) an immunoglobulin light chain CDR2 (CDR-L2) amino comprising the amino acid sequence set forth in SEQ ID NO: 75; and/or (f) and an immunoglobulin light chain CDR3 (CDR-L3) comprising the amino acid sequence set forth in SEQ ID NO: 76. In certain embodiments, the antibody that binds IGF1R comprises: (a) an immunoglobulin heavy chain CDR1 (CDR-H1) comprising the amino acid sequence set forth in SEQ ID NO: 81; (b) an immunoglobulin heavy chain CDR2 (CDR-H2) comprising the amino acid sequence set forth in SEQ ID NO: 82; (c) an immunoglobulin heavy chain CDR3 (CDR-H3) comprising the amino acid sequence set forth in SEQ ID NO: 83; (d) an immunoglobulin light chain CDR1 (CDR-L1) comprising the amino acid sequence set forth in SEQ ID NO: 84; (e) an immunoglobulin light chain CDR2 (CDR-L2) amino comprising the amino acid sequence set forth in SEQ ID NO: 85; and/or (f) and an immunoglobulin light chain CDR3 (CDR-L3) comprising the amino acid sequence set forth in SEQ ID NO: 86. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 7; and wherein the immunoglobulin light chain variable region comprises an amino acid sequence at least about 90%, 95%, 97%, 99%, or 100% identical to that set forth in SEQ ID NO: 8. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 27; and wherein the immunoglobulin light chain variable region comprises an amino acid sequence at least about 90%, 95%, 97%, 99%, or 100% identical to that set forth in SEQ ID NO: 28. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 37; and wherein the immunoglobulin light chain variable region comprises an amino acid sequence at least about 90%, 95%, 97%, 99%, or 100% identical to that set forth in SEQ ID NO: 38. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 47; and wherein the immunoglobulin light chain variable region comprises an amino acid sequence at least about 90%, 95%, 97%, 99%, or 100% identical to that set forth in SEQ ID NO: 48. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 57; and wherein the immunoglobulin light chain variable region comprises an amino acid sequence at least about 90%, 95%, 97%, 99%, or 100% identical to that set forth in SEQ ID NO: 58. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 67; and wherein the immunoglobulin light chain variable region comprises an amino acid sequence at least about 90%, 95%, 97%, 99%, or 100% identical to that set forth in SEQ ID NO: 68. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 77; and wherein the immunoglobulin light chain variable region comprises an amino acid sequence at least about 90%, 95%, 97%, 99%, or 100% identical to that set forth in SEQ ID NO: 78. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 87; and wherein the immunoglobulin light chain variable region comprises an amino acid sequence at least about 90%, 95%, 97%, 99%, or 100% identical to that set forth in SEQ ID NO: 88. In certain embodiments, the antibody that binds IGF1R comprises an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 9; and wherein the immunoglobulin light chain comprises an amino acid sequence at least about 90%, 95%, 97%, 99%, or 100% identical to that set forth in SEQ ID NO: 10. In certain embodiments, the antibody that binds IGF1R comprises teprotumumab. In certain embodiments, the antibody that binds IGF inhibits signaling through IGF1R. In certain embodiments, the antibody that binds IGF is administered at dose of about 5 mg/kg to about 50 mg/kg. In certain embodiments, the antibody that binds IGF1R is administered at dose of about 5 mg/kg to about 25 mg/kg. In certain embodiments, the antibody that binds IGF1R is administered at dose of about 10 mg/kg to about 20 mg/kg. In certain embodiments, the antibody that binds IGF1R is administered at dose of about 10 mg/kg. In certain embodiments, the antibody that binds IGF1R is administered at dose of about 20 mg/kg. In certain embodiments, the antibody that binds IGF1R is administered at first dose of about 10 mg/kg and at a subsequent dose of about 20 mg/kg. In certain embodiments, the antibody that binds IGF1R is administered once every three weeks. In certain embodiments, the IGF1R inhibitor is included in a pharmaceutical formulation comprising a pharmaceutically acceptable excipient, carrier, or diluent. In certain embodiments, the pharmaceutical formulation is formulated for intravenous administration. In certain embodiments, the pharmaceutical formulation is formulated for subcutaneous administration. In certain embodiments, the pharmaceutical formulation is the IGF1R inhibitor reduces proptosis a patient afflicted with chronic TED. In certain embodiments, the IGF1R inhibitor reduces proptosis by at least about 1 mm in the patient afflicted with chronic TED. In certain embodiments, the IGF1R inhibitor reduces proptosis by at least about 2 mm in the patient afflicted with chronic TED. In certain embodiments, the IGF1R inhibitor reduces proptosis by at least about 3 mm in the patient afflicted with chronic TED. In certain embodiments, the IGF1R inhibitor reduces proptosis by at least about 4 mm in the patient afflicted with chronic TED. In certain embodiments, the IGF1R inhibitor reduces diplopia in a patient afflicted with chronic TED.

Described herein in one aspect is a method of treating an individual with inactive thyroid eye disease, the method comprising administering to an individual with inactive or chronic thyroid eye disease (TED) an insulin-like growth factor 1 receptor (IGF1R) inhibitor, thereby treating the inactive or chronic TED, wherein the IGF1R inhibitor is not teprotumumab. In certain embodiments, the inactive TED is associated with Grave's disease. In certain embodiments, the inactive TED has a Clinical Activity Score (CAS) of 1 or less. In certain embodiments, the inactive or chronic TED has a Clinical Activity Score (CAS) of 0. In certain embodiments, the inactive TED is chronic TED. In certain embodiments, the chronic TED has been inactive for at least 6 months prior to treatment. In certain embodiments, the inactive or chronic TED has been inactive or chronic for at least 12 months prior to treatment. In certain embodiments, the inactive or chronic TED has been previously treated with an IGF1R inhibitor. In certain embodiments, the IGF1R inhibitor comprises ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof. In certain embodiments, the IGF1R inhibitor comprises an antibody that binds IGF1R. In certain embodiments, the antibody that binds IGF1R is chimeric or humanized. In certain embodiments, the antibody that binds IGF1R is an IgG antibody. In certain embodiments, the antibody that binds IGF1R is a Fab, F(ab)₂, a single-domain antibody, or a single chain variable fragment (scFv). In certain embodiments, the IGF1R inhibitor is an antibody comprising ganitumab, figitumumab, MEDI-573, cixutumumab, dalotuzumab, robatumumab, robatumumab, AVE1642, BIIB022, xentuzumab, istiratumab, or any combination thereof. In certain embodiments, the antibody that binds IGF1R comprises: (a) an immunoglobulin heavy chain CDR1 (CDR-H1) comprising the amino acid sequence set forth in any one of SEQ ID NO: 11, 21, 31, 41, 51, 61, 71, or 81; (b) an immunoglobulin heavy chain CDR2 (CDR-H2) comprising the amino acid sequence set forth in any one of SEQ ID NO: 12, 22, 32, 42, 52, 62, 72, or 82; (c) an immunoglobulin heavy chain CDR3 (CDR-H3) comprising the amino acid sequence set forth in any one of SEQ ID NO: 13, 23, 33, 43, 53, 63, 73, or 83; (d) an immunoglobulin light chain CDR1 (CDR-L1) comprising the amino acid sequence set forth in any one of SEQ ID NO: 14, 24, 34, 44, 54, 64, 74, or 84; (e) an immunoglobulin light chain CDR2 (CDR-L2) amino comprising the amino acid sequence set forth in any one of SEQ ID NO: 15, 25, 35, 45, 55, 65, 75, or 85; and/or (f) and an immunoglobulin light chain CDR3 (CDR-L3) comprising the amino acid sequence set forth in any one of SEQ ID NO: 16, 26, 36, 46, 56, 66, 76, or 86. In certain embodiments, the antibody that binds IGF1R comprises: (a) an immunoglobulin heavy chain CDR1 (CDR-H1) comprising the amino acid sequence set forth in SEQ ID NO: 11; (b) an immunoglobulin heavy chain CDR2 (CDR-H2) comprising the amino acid sequence set forth in SEQ ID NO: 12; (c) an immunoglobulin heavy chain CDR3 (CDR-H3) comprising the amino acid sequence set forth in SEQ ID NO: 13; (d) an immunoglobulin light chain CDR1 (CDR-L1) comprising the amino acid sequence set forth in SEQ ID NO: 14; (e) an immunoglobulin light chain CDR2 (CDR-L2) amino comprising the amino acid sequence set forth in SEQ ID NO: 15; and/or (f) and an immunoglobulin light chain CDR3 (CDR-L3) comprising the amino acid sequence set forth in SEQ ID NO: 16. In certain embodiments, the antibody that binds IGF1R comprises: (a) an immunoglobulin heavy chain CDR1 (CDR-H1) comprising the amino acid sequence set forth in SEQ ID NO: 21; (b) an immunoglobulin heavy chain CDR2 (CDR-H2) comprising the amino acid sequence set forth in SEQ ID NO: 22; (c) an immunoglobulin heavy chain CDR3 (CDR-H3) comprising the amino acid sequence set forth in SEQ ID NO: 23; (d) an immunoglobulin light chain CDR1 (CDR-L1) comprising the amino acid sequence set forth in SEQ ID NO: 24; (e) an immunoglobulin light chain CDR2 (CDR-L2) amino comprising the amino acid sequence set forth in SEQ ID NO: 25; and/or (f) and an immunoglobulin light chain CDR3 (CDR-L3) comprising the amino acid sequence set forth in SEQ ID NO: 26. In certain embodiments, the antibody that binds IGF1R comprises: (a) an immunoglobulin heavy chain CDR1 (CDR-H1) comprising the amino acid sequence set forth in SEQ ID NO: 31; (b) an immunoglobulin heavy chain CDR2 (CDR-H2) comprising the amino acid sequence set forth in SEQ ID NO: 32; (c) an immunoglobulin heavy chain CDR3 (CDR-H3) comprising the amino acid sequence set forth in SEQ ID NO: 33; (d) an immunoglobulin light chain CDR1 (CDR-L1) comprising the amino acid sequence set forth in SEQ ID NO: 34; (e) an immunoglobulin light chain CDR2 (CDR-L2) amino comprising the amino acid sequence set forth in SEQ ID NO: 35; and/or (f) and an immunoglobulin light chain CDR3 (CDR-L3) comprising the amino acid sequence set forth in SEQ ID NO: 36. In certain embodiments, the antibody that binds IGF1R comprises: (a) an immunoglobulin heavy chain CDR1 (CDR-H1) comprising the amino acid sequence set forth in SEQ ID NO: 41; (b) an immunoglobulin heavy chain CDR2 (CDR-H2) comprising the amino acid sequence set forth in SEQ ID NO: 42; (c) an immunoglobulin heavy chain CDR3 (CDR-H3) comprising the amino acid sequence set forth in SEQ ID NO: 43; (d) an immunoglobulin light chain CDR1 (CDR-L1) comprising the amino acid sequence set forth in SEQ ID NO: 44; (e) an immunoglobulin light chain CDR2 (CDR-L2) amino comprising the amino acid sequence set forth in SEQ ID NO: 45; and/or (f) and an immunoglobulin light chain CDR3 (CDR-L3) comprising the amino acid sequence set forth in SEQ ID NO: 46. In certain embodiments, the antibody that binds IGF1R comprises: (a) an immunoglobulin heavy chain CDR1 (CDR-H1) comprising the amino acid sequence set forth in SEQ ID NO: 51; (b) an immunoglobulin heavy chain CDR2 (CDR-H2) comprising the amino acid sequence set forth in SEQ ID NO: 52; (c) an immunoglobulin heavy chain CDR3 (CDR-H3) comprising the amino acid sequence set forth in SEQ ID NO: 53; (d) an immunoglobulin light chain CDR1 (CDR-L1) comprising the amino acid sequence set forth in SEQ ID NO: 54; (e) an immunoglobulin light chain CDR2 (CDR-L2) amino comprising the amino acid sequence set forth in SEQ ID NO: 55; and/or (f) and an immunoglobulin light chain CDR3 (CDR-L3) comprising the amino acid sequence set forth in SEQ ID NO: 56. In certain embodiments, the antibody that binds IGF1R comprises: (a) an immunoglobulin heavy chain CDR1 (CDR-H1) comprising the amino acid sequence set forth in SEQ ID NO: 61; (b) an immunoglobulin heavy chain CDR2 (CDR-H2) comprising the amino acid sequence set forth in SEQ ID NO: 62; (c) an immunoglobulin heavy chain CDR3 (CDR-H3) comprising the amino acid sequence set forth in SEQ ID NO: 63; (d) an immunoglobulin light chain CDR1 (CDR-L1) comprising the amino acid sequence set forth in SEQ ID NO: 64; (e) an immunoglobulin light chain CDR2 (CDR-L2) amino comprising the amino acid sequence set forth in SEQ ID NO: 65; and/or (f) and an immunoglobulin light chain CDR3 (CDR-L3) comprising the amino acid sequence set forth in SEQ ID NO: 66. In certain embodiments, the antibody that binds IGF1R comprises: (a) an immunoglobulin heavy chain CDR1 (CDR-H1) comprising the amino acid sequence set forth in SEQ ID NO: 71; (b) an immunoglobulin heavy chain CDR2 (CDR-H2) comprising the amino acid sequence set forth in SEQ ID NO: 72; (c) an immunoglobulin heavy chain CDR3 (CDR-H3) comprising the amino acid sequence set forth in SEQ ID NO: 73; (d) an immunoglobulin light chain CDR1 (CDR-L1) comprising the amino acid sequence set forth in SEQ ID NO: 74; (e) an immunoglobulin light chain CDR2 (CDR-L2) amino comprising the amino acid sequence set forth in SEQ ID NO: 75; and/or (f) and an immunoglobulin light chain CDR3 (CDR-L3) comprising the amino acid sequence set forth in SEQ ID NO: 76. In certain embodiments, the antibody that binds IGF1R comprises: (a) an immunoglobulin heavy chain CDR1 (CDR-H1) comprising the amino acid sequence set forth in SEQ ID NO: 81; (b) an immunoglobulin heavy chain CDR2 (CDR-H2) comprising the amino acid sequence set forth in SEQ ID NO: 82; (c) an immunoglobulin heavy chain CDR3 (CDR-H3) comprising the amino acid sequence set forth in SEQ ID NO: 83; (d) an immunoglobulin light chain CDR1 (CDR-L1) comprising the amino acid sequence set forth in SEQ ID NO: 84; (e) an immunoglobulin light chain CDR2 (CDR-L2) amino comprising the amino acid sequence set forth in SEQ ID NO: 85; and/or (f) and an immunoglobulin light chain CDR3 (CDR-L3) comprising the amino acid sequence set forth in SEQ ID NO: 86. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 27; and wherein the immunoglobulin light chain variable region comprises an amino acid sequence at least about 90%, 95%, 97%, 99%, or 100% identical to that set forth in SEQ ID NO: 28. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 37; and wherein the immunoglobulin light chain variable region comprises an amino acid sequence at least about 90%, 95%, 97%, 99%, or 100% identical to that set forth in SEQ ID NO: 38. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 47; and wherein the immunoglobulin light chain variable region comprises an amino acid sequence at least about 90%, 95%, 97%, 99%, or 100% identical to that set forth in SEQ ID NO: 48. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 57; and wherein the immunoglobulin light chain variable region comprises an amino acid sequence at least about 90%, 95%, 97%, 99%, or 100% identical to that set forth in SEQ ID NO: 58. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 67; and wherein the immunoglobulin light chain variable region comprises an amino acid sequence at least about 90%, 95%, 97%, 99%, or 100% identical to that set forth in SEQ ID NO: 68. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 77; and wherein the immunoglobulin light chain variable region comprises an amino acid sequence at least about 90%, 95%, 97%, 99%, or 100% identical to that set forth in SEQ ID NO: 78. In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 87; and wherein the immunoglobulin light chain variable region comprises an amino acid sequence at least about 90%, 95%, 97%, 99%, or 100% identical to that set forth in SEQ ID NO: 88. In certain embodiments, the antibody that binds IGF1R comprises an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 9; and wherein the immunoglobulin light chain comprises an amino acid sequence at least about 90%, 95%, 97%, 99%, or 100% identical to that set forth in SEQ ID NO: 10. In certain embodiments, the antibody that binds IGF1R inhibits signaling through IGF1R. In certain embodiments, the antibody that binds IGF1R is administered at dose of about 5 mg/kg to about 50 mg/kg. In certain embodiments, the antibody that binds IGF1R is administered at dose of about 5 mg/kg to about 25 mg/kg. In certain embodiments, the antibody that binds IGF1R is administered at dose of about 10 mg/kg to about 20 mg/kg. In certain embodiments, the antibody that binds IGF1R is administered at dose of about 10 mg/kg. In certain embodiments, the antibody that binds IGF1R is administered at dose of about 20 mg/kg. In certain embodiments, the antibody that binds IGF1R is administered at first dose of about 10 mg/kg and at a subsequent dose of about 20 mg/kg. In certain embodiments, the antibody that binds IGF1R is administered once every three weeks. In certain embodiments, the IGF1R inhibitor is included in a pharmaceutical formulation comprising a pharmaceutically acceptable excipient, carrier, or diluent. In certain embodiments, the pharmaceutical formulation is formulated for intravenous administration. In certain embodiments, the pharmaceutical formulation is formulated for subcutaneous administration. In certain embodiments, the pharmaceutical formulation is the IGF1R inhibitor reduces proptosis a patient afflicted with chronic TED. In certain embodiments, the IGF1R inhibitor reduces proptosis by at least about 1 mm in the patient afflicted with chronic TED. In certain embodiments, the IGF1R inhibitor reduces proptosis by at least about 2 mm in the patient afflicted with chronic TED. In certain embodiments, the IGF1R inhibitor reduces proptosis by at least about 3 mm in the patient afflicted with chronic TED. In certain embodiments, the IGF1R inhibitor reduces proptosis by at least about 4 mm in the patient afflicted with chronic TED. In certain embodiments, the IGF1R inhibitor reduces diplopia in a patient afflicted with chronic TED.

In some cases, the IGF1R inhibitor reduces diplopia in the patient afflicted with chronic TED. In some cases, the IGF1R inhibitor reduces a diplopia score in the patient afflicted with chronic TED by 3 grades. In some cases, the IGF1R inhibitor reduces a diplopia score in the patient afflicted with chronic TED by at least 2 grades. In some cases, the IGF1R inhibitor reduces a diplopia score in the patient afflicted with chronic TED by at least 1 grade. In some cases, the IGF1R inhibitor reduces a diplopia score in the patient afflicted with chronic TED to 0.

In some cases, the IGF1R inhibitor reduces binocular diplopia in the patient afflicted with chronic TED. In some cases, the IGF1R inhibitor reduces a binocular diplopia score in the patient afflicted with chronic TED by 3 grades. In some cases, the IGF1R inhibitor reduces a binocular diplopia score in the patient afflicted with chronic TED by at least 2 grades. In some cases, the IGF1R inhibitor reduces a binocular diplopia score in the patient afflicted with chronic TED by at least 1 grade. In some cases, the IGF1R inhibitor reduces a binocular diplopia score in the patient afflicted with chronic TED to 0.

In an aspect, described herein is a method of treating an individual with thyroid eye disease (TED), the method comprising administering teprotumumab to the individual with TED, wherein the individual with TED has a Clinical Activity Score (CAS) of less than 2 for 6 months or longer. In an aspect, described herein is a method of treating an individual with thyroid eye disease (TED), the method comprising administering teprotumumab to the individual with TED, wherein the individual with TED has a CAS of less than 2 for 7 months or longer. In an aspect, described herein is a method of treating an individual with thyroid eye disease (TED), the method comprising administering teprotumumab to the individual with TED, wherein the individual with TED has a CAS of less than 2 for 8 months or longer. In an aspect, described herein is a method of treating an individual with thyroid eye disease (TED), the method comprising administering teprotumumab to the individual with TED, wherein the individual with TED has a CAS of less than 2 for 9 months or longer. In an aspect, described herein is a method of treating an individual with thyroid eye disease (TED), the method comprising administering teprotumumab to the individual with TED, wherein the individual with TED has a CAS of less than 2 for 10 months or longer. In an aspect, described herein is a method of treating an individual with thyroid eye disease (TED), the method comprising administering teprotumumab to the individual with TED, wherein the individual with TED has a CAS of less than 2 for 11 months or longer. In an aspect, described herein is a method of treating an individual with thyroid eye disease (TED), the method comprising administering teprotumumab to the individual with TED, wherein the individual with TED has a CAS of less than 2 for 12 months or longer. In an aspect, described herein is a method of treating an individual with thyroid eye disease (TED), the method comprising administering teprotumumab to the individual with TED, wherein the individual with TED has a CAS of less than 2 for 13 months or longer. In an aspect, described herein is a method of treating an individual with thyroid eye disease (TED), the method comprising administering teprotumumab to the individual with TED, wherein the individual with TED has a CAS of less than 2 for 14 months or longer. In an aspect, described herein is a method of treating an individual with thyroid eye disease (TED), the method comprising administering teprotumumab to the individual with TED, wherein the individual with TED has a CAS of less than 2 for 15 months or longer. In an aspect, described herein is a method of treating an individual with thyroid eye disease (TED), the method comprising administering teprotumumab to the individual with TED, wherein the individual with TED has a CAS of less than 2 for 16 months or longer. In an aspect, described herein is a method of treating an individual with thyroid eye disease (TED), the method comprising administering teprotumumab to the individual with TED, wherein the individual with TED has a CAS of less than 2 for 17 months or longer. In an aspect, described herein is a method of treating an individual with thyroid eye disease (TED), the method comprising administering teprotumumab to the individual with TED, wherein the individual with TED has a CAS of less than 2 for 18 months or longer. In an aspect, described herein is a method of treating an individual with thyroid eye disease (TED), the method comprising administering teprotumumab to the individual with TED, wherein the individual with TED has a CAS of less than 2 for 19 months or longer. In an aspect, described herein is a method of treating an individual with thyroid eye disease (TED), the method comprising administering teprotumumab to the individual with TED, wherein the individual with TED has a CAS of less than 2 for 20 months or longer. In an aspect, described herein is a method of treating an individual with thyroid eye disease (TED), the method comprising administering teprotumumab to the individual with TED, wherein the individual with TED has a CAS of less than 2 for 21 months or longer. In an aspect, described herein is a method of treating an individual with thyroid eye disease (TED), the method comprising administering teprotumumab to the individual with TED, wherein the individual with TED has a CAS of less than 2 for 22 months or longer. In an aspect, described herein is a method of treating an individual with thyroid eye disease (TED), the method comprising administering teprotumumab to the individual with TED, wherein the individual with TED has a CAS of less than 2 for 23 months or longer. In an aspect, described herein is a method of treating an individual with thyroid eye disease (TED), the method comprising administering teprotumumab to the individual with TED, wherein the individual with TED has a CAS of less than 2 for 24 months or longer. In an aspect, described herein is a method of treating an individual with thyroid eye disease (TED), the method comprising administering teprotumumab to the individual with TED, wherein the individual with TED has a CAS of less than 2 for 25 months or longer. In an aspect, described herein is a method of treating an individual with thyroid eye disease (TED), the method comprising administering teprotumumab to the individual with TED, wherein the individual with TED has a CAS of less than 2 for 26 months or longer. In an aspect, described herein is a method of treating an individual with thyroid eye disease (TED), the method comprising administering teprotumumab to the individual with TED, wherein the individual with TED has a CAS of less than 2 for 27 months or longer. In an aspect, described herein is a method of treating an individual with thyroid eye disease (TED), the method comprising administering teprotumumab to the individual with TED, wherein the individual with TED has a CAS of less than 2 for 28 months or longer. In an aspect, described herein is a method of treating an individual with thyroid eye disease (TED), the method comprising administering teprotumumab to the individual with TED, wherein the individual with TED has a CAS of less than 2 for 29 months or longer. In an aspect, described herein is a method of treating an individual with thyroid eye disease (TED), the method comprising administering teprotumumab to the individual with TED, wherein the individual with TED has a CAS of less than 2 for 30 months or longer. In an aspect, described herein is a method of treating an individual with thyroid eye disease (TED), the method comprising administering teprotumumab to the individual with TED, wherein the individual with TED has a CAS of less than 2 for 31 months or longer. In an aspect, described herein is a method of treating an individual with thyroid eye disease (TED), the method comprising administering teprotumumab to the individual with TED, wherein the individual with TED has a CAS of less than 2 for 32 months or longer. In an aspect, described herein is a method of treating an individual with thyroid eye disease (TED), the method comprising administering teprotumumab to the individual with TED, wherein the individual with TED has a CAS of less than 2 for 33 months or longer. In an aspect, described herein is a method of treating an individual with thyroid eye disease (TED), the method comprising administering teprotumumab to the individual with TED, wherein the individual with TED has a CAS of less than 2 for 34 months or longer. In an aspect, described herein is a method of treating an individual with thyroid eye disease (TED), the method comprising administering teprotumumab to the individual with TED, wherein the individual with TED has a CAS of less than 2 for 35 months or longer. In an aspect, described herein is a method of treating an individual with thyroid eye disease (TED), the method comprising administering teprotumumab to the individual with TED, wherein the individual with TED has a CAS of less than 2 for 36 months or longer.

In an aspect, described herein is a method of treating an individual with thyroid eye disease (TED), the method comprising administering teprotumumab to the individual with TED, wherein the individual with TED has a CAS of less than 1 for 6 months or longer. In an aspect, described herein is a method of treating an individual with thyroid eye disease (TED), the method comprising administering teprotumumab to the individual with TED, wherein the individual with TED has a CAS of less than 1 for 7 months or longer. In an aspect, described herein is a method of treating an individual with thyroid eye disease (TED), the method comprising administering teprotumumab to the individual with TED, wherein the individual with TED has a CAS of less than 1 for 8 months or longer. In an aspect, described herein is a method of treating an individual with thyroid eye disease (TED), the method comprising administering teprotumumab to the individual with TED, wherein the individual with TED has a CAS of less than 1 for 9 months or longer. In an aspect, described herein is a method of treating an individual with thyroid eye disease (TED), the method comprising administering teprotumumab to the individual with TED, wherein the individual with TED has a CAS of less than 1 for 10 months or longer. In an aspect, described herein is a method of treating an individual with thyroid eye disease (TED), the method comprising administering teprotumumab to the individual with TED, wherein the individual with TED has a CAS of less than 1 for 11 months or longer. In an aspect, described herein is a method of treating an individual with thyroid eye disease (TED), the method comprising administering teprotumumab to the individual with TED, wherein the individual with TED has a CAS of less than 1 for 12 months or longer. In an aspect, described herein is a method of treating an individual with thyroid eye disease (TED), the method comprising administering teprotumumab to the individual with TED, wherein the individual with TED has a CAS of less than 1 for 13 months or longer. In an aspect, described herein is a method of treating an individual with thyroid eye disease (TED), the method comprising administering teprotumumab to the individual with TED, wherein the individual with TED has a CAS of less than 1 for 14 months or longer. In an aspect, described herein is a method of treating an individual with thyroid eye disease (TED), the method comprising administering teprotumumab to the individual with TED, wherein the individual with TED has a CAS of less than 1 for 15 months or longer. In an aspect, described herein is a method of treating an individual with thyroid eye disease (TED), the method comprising administering teprotumumab to the individual with TED, wherein the individual with TED has a CAS of less than 1 for 16 months or longer. In an aspect, described herein is a method of treating an individual with thyroid eye disease (TED), the method comprising administering teprotumumab to the individual with TED, wherein the individual with TED has a CAS of less than 1 for 17 months or longer. In an aspect, described herein is a method of treating an individual with thyroid eye disease (TED), the method comprising administering teprotumumab to the individual with TED, wherein the individual with TED has a CAS of less than 1 for 18 months or longer. In an aspect, described herein is a method of treating an individual with thyroid eye disease (TED), the method comprising administering teprotumumab to the individual with TED, wherein the individual with TED has a CAS of less than 1 for 19 months or longer. In an aspect, described herein is a method of treating an individual with thyroid eye disease (TED), the method comprising administering teprotumumab to the individual with TED, wherein the individual with TED has a CAS of less than 1 for 20 months or longer. In an aspect, described herein is a method of treating an individual with thyroid eye disease (TED), the method comprising administering teprotumumab to the individual with TED, wherein the individual with TED has a CAS of less than 1 for 21 months or longer. In an aspect, described herein is a method of treating an individual with thyroid eye disease (TED), the method comprising administering teprotumumab to the individual with TED, wherein the individual with TED has a CAS of less than 1 for 22 months or longer. In an aspect, described herein is a method of treating an individual with thyroid eye disease (TED), the method comprising administering teprotumumab to the individual with TED, wherein the individual with TED has a CAS of less than 1 for 23 months or longer. In an aspect, described herein is a method of treating an individual with thyroid eye disease (TED), the method comprising administering teprotumumab to the individual with TED, wherein the individual with TED has a CAS of less than 1 for 24 months or longer. In an aspect, described herein is a method of treating an individual with thyroid eye disease (TED), the method comprising administering teprotumumab to the individual with TED, wherein the individual with TED has a CAS of less than 1 for 25 months or longer. In an aspect, described herein is a method of treating an individual with thyroid eye disease (TED), the method comprising administering teprotumumab to the individual with TED, wherein the individual with TED has a CAS of less than 1 for 26 months or longer. In an aspect, described herein is a method of treating an individual with thyroid eye disease (TED), the method comprising administering teprotumumab to the individual with TED, wherein the individual with TED has a CAS of less than 1 for 27 months or longer. In an aspect, described herein is a method of treating an individual with thyroid eye disease (TED), the method comprising administering teprotumumab to the individual with TED, wherein the individual with TED has a CAS of less than 1 for 28 months or longer. In an aspect, described herein is a method of treating an individual with thyroid eye disease (TED), the method comprising administering teprotumumab to the individual with TED, wherein the individual with TED has a CAS of less than 1 for 29 months or longer. In an aspect, described herein is a method of treating an individual with thyroid eye disease (TED), the method comprising administering teprotumumab to the individual with TED, wherein the individual with TED has a CAS of less than 1 for 30 months or longer. In an aspect, described herein is a method of treating an individual with thyroid eye disease (TED), the method comprising administering teprotumumab to the individual with TED, wherein the individual with TED has a CAS of less than 1 for 31 months or longer. In an aspect, described herein is a method of treating an individual with thyroid eye disease (TED), the method comprising administering teprotumumab to the individual with TED, wherein the individual with TED has a CAS of less than 1 for 32 months or longer. In an aspect, described herein is a method of treating an individual with thyroid eye disease (TED), the method comprising administering teprotumumab to the individual with TED, wherein the individual with TED has a CAS of less than 1 for 33 months or longer. In an aspect, described herein is a method of treating an individual with thyroid eye disease (TED), the method comprising administering teprotumumab to the individual with TED, wherein the individual with TED has a CAS of less than 1 for 34 months or longer. In an aspect, described herein is a method of treating an individual with thyroid eye disease (TED), the method comprising administering teprotumumab to the individual with TED, wherein the individual with TED has a CAS of less than 1 for 35 months or longer. In an aspect, described herein is a method of treating an individual with thyroid eye disease (TED), the method comprising administering teprotumumab to the individual with TED, wherein the individual with TED has a CAS of less than 1 for 36 months or longer.

In an aspect, described herein is a method of treating an individual with thyroid eye disease (TED), the method comprising administering teprotumumab to the individual with TED, wherein the individual with TED has a CAS of 0 for 6 months or longer. In an aspect, described herein is a method of treating an individual with thyroid eye disease (TED), the method comprising administering teprotumumab to the individual with TED, wherein the individual with TED has a CAS of 0 for 7 months or longer. In an aspect, described herein is a method of treating an individual with thyroid eye disease (TED), the method comprising administering teprotumumab to the individual with TED, wherein the individual with TED has a CAS of 0 for 8 months or longer. In an aspect, described herein is a method of treating an individual with thyroid eye disease (TED), the method comprising administering teprotumumab to the individual with TED, wherein the individual with TED has a CAS of 0 for 9 months or longer. In an aspect, described herein is a method of treating an individual with thyroid eye disease (TED), the method comprising administering teprotumumab to the individual with TED, wherein the individual with TED has a CAS of 0 for 10 months or longer. In an aspect, described herein is a method of treating an individual with thyroid eye disease (TED), the method comprising administering teprotumumab to the individual with TED, wherein the individual with TED has a CAS of 0 for 11 months or longer. In an aspect, described herein is a method of treating an individual with thyroid eye disease (TED), the method comprising administering teprotumumab to the individual with TED, wherein the individual with TED has a CAS of 0 for 12 months or longer. In an aspect, described herein is a method of treating an individual with thyroid eye disease (TED), the method comprising administering teprotumumab to the individual with TED, wherein the individual with TED has a CAS of 0 for 13 months or longer. In an aspect, described herein is a method of treating an individual with thyroid eye disease (TED), the method comprising administering teprotumumab to the individual with TED, wherein the individual with TED has a CAS of 0 for 14 months or longer. In an aspect, described herein is a method of treating an individual with thyroid eye disease (TED), the method comprising administering teprotumumab to the individual with TED, wherein the individual with TED has a CAS of 0 for 15 months or longer. In an aspect, described herein is a method of treating an individual with thyroid eye disease (TED), the method comprising administering teprotumumab to the individual with TED, wherein the individual with TED has a CAS of 0 for 16 months or longer. In an aspect, described herein is a method of treating an individual with thyroid eye disease (TED), the method comprising administering teprotumumab to the individual with TED, wherein the individual with TED has a CAS of 0 for 17 months or longer. In an aspect, described herein is a method of treating an individual with thyroid eye disease (TED), the method comprising administering teprotumumab to the individual with TED, wherein the individual with TED has a CAS of 0 for 18 months or longer. In an aspect, described herein is a method of treating an individual with thyroid eye disease (TED), the method comprising administering teprotumumab to the individual with TED, wherein the individual with TED has a CAS of 0 for 19 months or longer. In an aspect, described herein is a method of treating an individual with thyroid eye disease (TED), the method comprising administering teprotumumab to the individual with TED, wherein the individual with TED has a CAS of 0 for 20 months or longer. In an aspect, described herein is a method of treating an individual with thyroid eye disease (TED), the method comprising administering teprotumumab to the individual with TED, wherein the individual with TED has a CAS of 0 for 21 months or longer. In an aspect, described herein is a method of treating an individual with thyroid eye disease (TED), the method comprising administering teprotumumab to the individual with TED, wherein the individual with TED has a CAS of 0 for 22 months or longer. In an aspect, described herein is a method of treating an individual with thyroid eye disease (TED), the method comprising administering teprotumumab to the individual with TED, wherein the individual with TED has a CAS of 0 for 23 months or longer. In an aspect, described herein is a method of treating an individual with thyroid eye disease (TED), the method comprising administering teprotumumab to the individual with TED, wherein the individual with TED has a CAS of 0 for 24 months or longer. In an aspect, described herein is a method of treating an individual with thyroid eye disease (TED), the method comprising administering teprotumumab to the individual with TED, wherein the individual with TED has a CAS of 0 for 25 months or longer. In an aspect, described herein is a method of treating an individual with thyroid eye disease (TED), the method comprising administering teprotumumab to the individual with TED, wherein the individual with TED has a CAS of 0 for 26 months or longer. In an aspect, described herein is a method of treating an individual with thyroid eye disease (TED), the method comprising administering teprotumumab to the individual with TED, wherein the individual with TED has a CAS of 0 for 27 months or longer. In an aspect, described herein is a method of treating an individual with thyroid eye disease (TED), the method comprising administering teprotumumab to the individual with TED, wherein the individual with TED has a CAS of 0 for 28 months or longer. In an aspect, described herein is a method of treating an individual with thyroid eye disease (TED), the method comprising administering teprotumumab to the individual with TED, wherein the individual with TED has a CAS of 0 for 29 months or longer. In an aspect, described herein is a method of treating an individual with thyroid eye disease (TED), the method comprising administering teprotumumab to the individual with TED, wherein the individual with TED has a CAS of 0 for 30 months or longer. In an aspect, described herein is a method of treating an individual with thyroid eye disease (TED), the method comprising administering teprotumumab to the individual with TED, wherein the individual with TED has a CAS of 0 for 31 months or longer. In an aspect, described herein is a method of treating an individual with thyroid eye disease (TED), the method comprising administering teprotumumab to the individual with TED, wherein the individual with TED has a CAS of 0 for 32 months or longer. In an aspect, described herein is a method of treating an individual with thyroid eye disease (TED), the method comprising administering teprotumumab to the individual with TED, wherein the individual with TED has a CAS of 0 for 33 months or longer. In an aspect, described herein is a method of treating an individual with thyroid eye disease (TED), the method comprising administering teprotumumab to the individual with TED, wherein the individual with TED has a CAS of 0 for 34 months or longer. In an aspect, described herein is a method of treating an individual with thyroid eye disease (TED), the method comprising administering teprotumumab to the individual with TED, wherein the individual with TED has a CAS of 0 for 35 months or longer. In an aspect, described herein is a method of treating an individual with thyroid eye disease (TED), the method comprising administering teprotumumab to the individual with TED, wherein the individual with TED has a CAS of 0 for 36 months or longer.

### BRIEF DESCRIPTIONS OF THE DRAWINGS

FIG. 1 illustrates patient disposition during a clinical trial for evaluating the efficacy and safety of teprotumumab (TEPEZZA®) in treating patients with chronic (inactive) thyroid eye disease (TED).

FIG. 2 illustrates the change in proptosis from baseline for patients with chronic (inactive) thyroid eye disease (TED) administered with either teprotumumab (TEPEZZA®) or a placebo.

FIG. 3 illustrates the proptosis responder rate for patients with chronic (inactive) thyroid eye disease (TED) administered with either teprotumumab (TEPEZZA®) or a placebo.

FIG. 4 illustrates the change in Graves' Ophthalmopathy Quality of Life (GO-QoL) Visual Function from for patients with chronic (inactive) thyroid eye disease (TED) administered with either teprotumumab (TEPEZZA®) or a placebo.

FIG. 5 illustrates the change in Graves' Ophthalmopathy Quality of Life (GO-QoL) Appearance from baseline for patients with chronic (inactive) thyroid eye disease (TED) administered with either teprotumumab (TEPEZZA®) or a placebo.

FIG. 6 illustrates the change in diplopia represented as response rate from baseline for patients with chronic (inactive) thyroid eye disease (TED) administered with either teprotumumab (TEPEZZA®) or a placebo.

FIG. 7 illustrates the diplopia responder rate and the complete responder rate for patients with chronic (inactive) thyroid eye disease (TED) administered with either teprotumumab (TEPEZZA®) or a placebo.

### DETAILED DESCRIPTION

Described herein, in certain embodiments, is a method of treating an individual with inactive thyroid eye disease (TED), the method comprising administering to an individual with inactive or chronic TED an insulin-like growth factor 1 receptor (IGF1R) inhibitor, thereby treating the inactive or chronic TED.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual”, “patient”, or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As used herein insulin-like growth factor 1 receptor or IGF1R refers to the receptor that binds insulin-like growth factor. IGF1R is present in all mammalian species. The amino acid sequence encoding human IGF1R can be found in the UniProt database (www.uniprot.org) as entry P08069. IGF1R can signal through a variety of signaling pathways, including but not limited to phosphoinositide 3-kinase/protein kinase B (PI3K/AKT) signaling and mitogen activated protein kinase (MAPK) signaling, to regulate cell proliferation and survival. In some embodiments described herein, an “insulin-like growth factor 1 receptor (IGF1R) inhibitor” refers to a compound, such as a small molecule or antibody, that inhibits IGF1R biological function (e.g., inhibition of MAPK signaling).

Among the provided antibodies are monoclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portions thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region.

The term “antibody” herein is used in the broadest sense and includes monoclonal antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

As used herein, the term TEPEZZA® refers to teprotumumab, and the terms may be used interchangeably.

For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma, a primary cell, or a library or heavy and light chain molecules and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this disclosure. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific target or antigen that shows significant increase in binding compared to an isotype control antibody.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, an antibody provided herein has a dissociation constant (KD) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target. In some embodiments, an antibody provided herein has a dissociation constant (KD) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antibody target. The antibody target can be IGF1R. KD can be measured by any suitable assay. In certain embodiments, KD can be measured using surface plasmon resonance assays (e.g., using a BIACORE®-2000, a BIACORE®-3000 or Octet).

In some embodiments, one or more amino acid modifications may be introduced into the Fc region of an antibody provided herein, thereby generating an Fc region variant. An Fc region herein is a C-terminal region of an immunoglobulin heavy chain that consists of the CH2 and CH3 domain of an Ig molecule. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antibodies described herein comprise the variable domains of the invention combined with constant domains comprising different Fc regions, selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIA_{H131}, and with lower affinity to FcγRIIA_{R131} FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1≥IgG3>>IgG4≅IgG2.

In some embodiments, the antibodies of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antibodies of this disclosure are variants that possess increased effector functions for applications in which increased immunogenicity would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due toits stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2(5):466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Treatment of Inactive or Chronic TED

Described herein are methods of treating inactive or chronic thyroid eye disease. Thyroid eye disease can be assessed by a clinical activity score (CAS) described by Mourits et al., British Journal of Ophthalmology, 1989, 73, no. 8, 639-644) as a way of assessing the degree of active disease. This score, based on the classical signs of acute inflammation (pain, redness, swelling, and impaired function), was proposed as a clinical classification to discriminate between active and inactive disease and was modified in 1997 (Mourits et al., Clinical Endocrinology, 1997. 47, no. 1, 9-14). CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present (1 point) or absent (0 points). The score at each efficacy assessment is the sum of all items present; giving a range of 0-7, where 0 or 1 constitutes inactive disease and 7 constitutes severe active ophthalmopathy. A CAS score can differentiate between active and inactive TED. Quality of life for individuals can be measured using self-assessment questionnaires such as with the Graves' Ophthalmopathy Quality of Life (GO-QoL) questionnaire. See e.g., Terwee C B, Gerding M N, Dekker F W, Prummel M F, Wiersinga W M. Development of a disease specific quality of life questionnaire for patients with Graves' ophthalmopathy: the GO-QoL. Br J Ophthalmol. 1998 July; 82(7):773-9.

Inactive TED refers to TED with a CAS score of less than 2. Chronic TED refers to TED that has been active or inactive for longer than 4 months. TED can be both inactive (a CAS of less than 2) and chronic (present for longer than 4 months).

In certain embodiments, described herein is a method of treating an individual with inactive or chronic thyroid eye disease, the method comprising administering to an individual with inactive or chronic thyroid eye disease (TED) an insulin-like growth factor 1 receptor (IGF1R) inhibitor, thereby treating the inactive or chronic TED. In certain embodiments, the individual with inactive or chronic TED has a CAS score of 1 or less. In certain embodiments, the individual with inactive or chronic TED has a CAS score of 0. In certain embodiments, the individual with inactive or chronic TED has previously been treated when exhibiting an active disease state. In certain embodiments, the TED has been inactive for at least 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, or 60 months. In certain embodiments, the TED has been chronic for at least 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, or 60 months or more. In certain embodiments, the TED has been chronic and inactive for at least 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, or 60 months. In certain embodiments, the individual with inactive or chronic TED has chronic TED.

The CAS score of an individual may be different in the different eyes of the individual. The methods used to treat TED may in certain instances be applied if both eyes have a CAS score of lower than 2, 1 or lower, or 0.

In certain embodiments, the methods described herein result in reduced proptosis, reduced diplopia, reduced orbital pain, reduced muscle volume, increased scores on the Graves' Ophthalmopathy Quality of Life (GO-QoL) questionnaire appearance and/or visual functioning subscales. In certain embodiments, the methods described herein result in reduced proptosis. In certain embodiments, the methods described herein result in reduced orbital pain. In certain embodiments, the methods described herein result in reduced muscle volume. In certain embodiments, the methods described herein result in increased scores on the Graves' Ophthalmopathy Quality of Life (GO-QoL) questionnaire appearance and/or visual functioning subscales. In certain embodiments, the methods described herein result in increased scores on the Graves' Ophthalmopathy Quality of Life (GO-QoL) questionnaire appearance and/or visual functioning subscales by at least 5, 6, 7, 8, 9, 10, 11, 12, 13, 14, 15, 16, 17, 18, 19, or 20 or more points.

In certain embodiments, the IGF1R inhibitor reduces proptosis a patient afflicted with chronic TED. In certain embodiments, the IGF1R inhibitor reduces proptosis by at least about 1 mm in the patient afflicted with chronic TED. In certain embodiments, the IGF1R inhibitor reduces proptosis by at least about 2 mm in the patient afflicted with chronic TED. In certain embodiments, the IGF1R inhibitor reduces proptosis by at least about 3 mm in the patient afflicted with chronic TED. In certain embodiments, the IGF1R inhibitor reduces proptosis by at least about 4 mm in the patient afflicted with chronic TED.

In some cases, the IGF1R inhibitor reduces diplopia by≥1 grade, ≥2 grades, or≥3 grades in a patient afflicted with chronic TED. In some cases, the IGF1R inhibitor reduces diplopia to a grade of 0 in a patient afflicted with chronic TED.

In certain embodiments, the IGF1R inhibitor reduces diplopia by 10%, 20%, 30%, 40%, 50% or more in a patient afflicted with chronic TED. In certain embodiments, the IGF1R inhibitor completely resolves diplopia

In certain embodiments, the IGF1R inhibitor reduces diplopia in a patient afflicted with chronic TED. In certain embodiments, the IGF1R inhibitor reduces diplopia by 10%, 20%, 30%, 40%, 50% or more in a patient afflicted with chronic TED. In certain embodiments, the IGF1R inhibitor completely resolves diplopia

In certain embodiments, the IGF1R inhibitor reduces binocular diplopia in a patient afflicted with chronic TED. Binocular diplopia occurs when both eyes are open and resolves when either eye is closed. It is caused by a misalignment of the eyes, also called strabismus.

Diplopia, including binocular diplopia, can be measured using a diplopia score that ranges from 0-3. A rating of 0 on the diplopia scale indicates no diplopia. A rating of 1 on the diplopia scale indicates intermittent diplopia (diplopia in primary position of gaze, when tired, or when first awakening). A rating of 2 on the diplopia scale indicates inconstant diplopia (diplopia at extremes of gaze). A rating of 3 indicates constant diplopia (continuous diplopia in primary or reading position). In some cases, a reduction of >1 grade indicates a patient is responsive.

In some cases, a binocular diplopia score is based on measurements taken from both eyes simultaneously. In some cases, a binocular diplopia score is based on measurements taken from each eye separately. In this latter case, the binocular diplopia score can be calculated based on the average score of both eyes.

In some cases, the IGF1R inhibitor reduces binocular diplopia by≥1 grade, ≥2 grades, or≥3 grades in a patient afflicted with chronic TED. In some cases, the IGF1R inhibitor reduces binocular diplopia to a grade of 0 in a patient afflicted with chronic TED.

In certain embodiments, the IGF1R inhibitor reduces binocular diplopia by 10%, 20%, 30%, 40%, 50% or more in a patient afflicted with chronic TED. In certain embodiments, the IGF1R inhibitor completely resolves binocular diplopia.

### IGF1R Inhibitors

IGF1R inhibitors and pharmaceutical compositions comprising IGF1R inhibitors can be used to treat TED in its inactive or chronic form.

In certain embodiments, the inhibitor of IGF1R biological function may exert its inhibitory function by binding to IGF1R. In certain embodiments, the inhibitor of IGF1R biological function may exert its inhibitory function by inhibiting IGF1R signaling. n certain embodiments, the inhibitor of IGF1R biological function may exert its inhibitory function by preventing IGF1R autophosphorylation. In certain embodiments, the inhibitor of IGF1R biological function may exert its inhibitory function by inhibiting IGF1R signaling by inhibiting signaling downstream of IGF1R. In certain embodiments, the inhibitor of IGF1R biological function may exert its inhibitory function in the presence of ligand (e.g., insulin or insulin-like growth factor).

In certain embodiments, the IGF1R inhibitor comprises ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof.

In certain embodiments, the IGF1R inhibitor for use in treating inactive or chronic TED comprises an antibody. In certain embodiments, the antibody comprises ganitumab, figitumumab, MEDI-573, cixutumumab, dalotuzumab, robatumumab, AVE1642, BIIB022, xentuzumab, istiratumab, and combinations thereof. In certain embodiments, the antibody comprises teprotumumab.

In certain embodiments, the antibody that binds IGF1R comprises: (a) an immunoglobulin heavy chain CDR1 (CDR-H1) comprising the amino acid sequence set forth in any one of SEQ ID NO: 1, 11, 21, 31, 41, 51, 61, 71, or 81; (b) an immunoglobulin heavy chain CDR2 (CDR-H2) comprising the amino acid sequence set forth in any one of SEQ ID NO: 2, 12, 22, 32, 42, 52, 62, 72, or 82; (c) an immunoglobulin heavy chain CDR3 (CDR-H3) comprising the amino acid sequence set forth in any one of SEQ ID NO: 3, 13, 23, 33, 43, 53, 63, 73, or 83; (d) an immunoglobulin light chain CDR1 (CDR-L1) comprising the amino acid sequence set forth in any one of SEQ ID NO: 4, 14, 24, 34, 44, 54, 64, 74, or 84; (e) an immunoglobulin light chain CDR2 (CDR-L2) amino comprising the amino acid sequence set forth in any one of SEQ ID NO: 5, 15, 25, 35, 45, 55, 65, 75, or 85; and/or (f) and an immunoglobulin light chain CDR3 (CDR-L3) comprising the amino acid sequence set forth in any one of SEQ ID NO: 6, 16, 26, 36, 46, 56, 66, 76, or 86.

In certain embodiments, the antibody that binds IGF1R comprises: (a) an immunoglobulin heavy chain CDR1 (CDR-H1) comprising the amino acid sequence set forth in SEQ ID NO: 1; (b) an immunoglobulin heavy chain CDR2 (CDR-H2) comprising the amino acid sequence set forth in SEQ ID NO: 2; (c) an immunoglobulin heavy chain CDR3 (CDR-H3) comprising the amino acid sequence set forth in SEQ ID NO: 3; (d) an immunoglobulin light chain CDR1 (CDR-L1) comprising the amino acid sequence set forth in SEQ ID NO: 4; (e) an immunoglobulin light chain CDR2 (CDR-L2) amino comprising the amino acid sequence set forth in SEQ ID NO: 5; and/or (f) and an immunoglobulin light chain CDR3 (CDR-L3) comprising the amino acid sequence set forth in SEQ ID NO: 6.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 7; and wherein the immunoglobulin light chain variable region comprises an amino acid sequence at least about 90%, 95%, 97%, 99%, or 100% identical to that set forth in SEQ ID NO: 8.

In certain embodiments, the antibody that binds IGF1R comprises an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 9; and wherein the immunoglobulin light chain comprises an amino acid sequence at least about 90%, 95%, 97%, 99%, or 100% identical to that set forth in SEQ ID NO: 10.

In certain embodiments, the antibody that binds IGF1R is teprotumumab.

In certain embodiments, the antibody that binds IGF1R comprises: (a) an immunoglobulin heavy chain CDR1 (CDR-H1) comprising the amino acid sequence set forth in SEQ ID NO: 11; (b) an immunoglobulin heavy chain CDR2 (CDR-H2) comprising the amino acid sequence set forth in SEQ ID NO: 12;(c) an immunoglobulin heavy chain CDR3 (CDR-H3) comprising the amino acid sequence set forth in SEQ ID NO: 13; (d) an immunoglobulin light chain CDR1 (CDR-L1) comprising the amino acid sequence set forth in SEQ ID NO: 14; (e) an immunoglobulin light chain CDR2 (CDR-L2) amino comprising the amino acid sequence set forth in SEQ ID NO: 15; and/or (f) and an immunoglobulin light chain CDR3 (CDR-L3) comprising the amino acid sequence set forth in SEQ ID NO: 16.

In certain embodiments the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18.

In certain embodiments, the antibody that binds IGF1R comprises: (a) an immunoglobulin heavy chain CDR1 (CDR-H1) comprising the amino acid sequence set forth in SEQ ID NO: 21; (b) an immunoglobulin heavy chain CDR2 (CDR-H2) comprising the amino acid sequence set forth in SEQ ID NO: 22; (c) an immunoglobulin heavy chain CDR3 (CDR-H3) comprising the amino acid sequence set forth in SEQ ID NO: 23; (d) an immunoglobulin light chain CDR1 (CDR-L1) comprising the amino acid sequence set forth in SEQ ID NO: 24; (e) an immunoglobulin light chain CDR2 (CDR-L2) amino comprising the amino acid sequence set forth in SEQ ID NO: 25; and/or (f) and an immunoglobulin light chain CDR3 (CDR-L3) comprising the amino acid sequence set forth in SEQ ID NO: 26.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 27; and wherein the immunoglobulin light chain variable region comprises an amino acid sequence at least about 90%, 95%, 97%, 99%, or 100% identical to that set forth in SEQ ID NO: 28.

In certain embodiments, the antibody that binds IGF1R comprises: (a) an immunoglobulin heavy chain CDR1 (CDR-H1) comprising the amino acid sequence set forth in SEQ ID NO: 31; (b) an immunoglobulin heavy chain CDR2 (CDR-H2) comprising the amino acid sequence set forth in SEQ ID NO: 32;(c) an immunoglobulin heavy chain CDR3 (CDR-H3) comprising the amino acid sequence set forth in SEQ ID NO: 33;(d) an immunoglobulin light chain CDR1 (CDR-L1) comprising the amino acid sequence set forth in SEQ ID NO: 34; (e) an immunoglobulin light chain CDR2 (CDR-L2) amino comprising the amino acid sequence set forth in SEQ ID NO: 35; and/or (f) and an immunoglobulin light chain CDR3 (CDR-L3) comprising the amino acid sequence set forth in SEQ ID NO: 36.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 37; and wherein the immunoglobulin light chain variable region comprises an amino acid sequence at least about 90%, 95%, 97%, 99%, or 100% identical to that set forth in SEQ ID NO: 38.

In certain embodiments, the antibody that binds IGF1R comprises: (a) an immunoglobulin heavy chain CDR1 (CDR-H1) comprising the amino acid sequence set forth in SEQ ID NO: 41; (b) an immunoglobulin heavy chain CDR2 (CDR-H2) comprising the amino acid sequence set forth in SEQ ID NO: 42;(c) an immunoglobulin heavy chain CDR3 (CDR-H3) comprising the amino acid sequence set forth in SEQ ID NO: 43;(d) an immunoglobulin light chain CDR1 (CDR-L1) comprising the amino acid sequence set forth in SEQ ID NO: 44; (e) an immunoglobulin light chain CDR2 (CDR-L2) amino comprising the amino acid sequence set forth in SEQ ID NO: 45; and/or (f) and an immunoglobulin light chain CDR3 (CDR-L3) comprising the amino acid sequence set forth in SEQ ID NO: 46.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 47; and wherein the immunoglobulin light chain variable region comprises an amino acid sequence at least about 90%, 95%, 97%, 99%, or 100% identical to that set forth in SEQ ID NO: 48.

In certain embodiments, the antibody that binds IGF1R comprises: (a)an immunoglobulin heavy chain CDR1 (CDR-H1) comprising the amino acid sequence set forth in SEQ ID NO: 51; (b) an immunoglobulin heavy chain CDR2 (CDR-H2) comprising the amino acid sequence set forth in SEQ ID NO: 52;(c) an immunoglobulin heavy chain CDR3 (CDR-H3) comprising the amino acid sequence set forth in SEQ ID NO: 53;(d) an immunoglobulin light chain CDR1 (CDR-L1) comprising the amino acid sequence set forth in SEQ ID NO: 54; (e) an immunoglobulin light chain CDR2 (CDR-L2) amino comprising the amino acid sequence set forth in SEQ ID NO: 55; and/or (f) and an immunoglobulin light chain CDR3 (CDR-L3) comprising the amino acid sequence set forth in SEQ ID NO: 56.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 57; and wherein the immunoglobulin light chain variable region comprises an amino acid sequence at least about 90%, 95%, 97%, 99%, or 100% identical to that set forth in SEQ ID NO: 58.

In certain embodiments, the antibody that binds IGF1R comprises: (a)an immunoglobulin heavy chain CDR1 (CDR-H1) comprising the amino acid sequence set forth in SEQ ID NO: 61; (b) an immunoglobulin heavy chain CDR2 (CDR-H2) comprising the amino acid sequence set forth in SEQ ID NO: 62;(c) an immunoglobulin heavy chain CDR3 (CDR-H3) comprising the amino acid sequence set forth in SEQ ID NO: 63;(d) an immunoglobulin light chain CDR1 (CDR-L1) comprising the amino acid sequence set forth in SEQ ID NO: 64;(e) an immunoglobulin light chain CDR2 (CDR-L2) amino comprising the amino acid sequence set forth in SEQ ID NO: 65; and/or (f) and an immunoglobulin light chain CDR3 (CDR-L3) comprising the amino acid sequence set forth in SEQ ID NO: 66.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 67; and wherein the immunoglobulin light chain variable region comprises an amino acid sequence at least about 90%, 95%, 97%, 99%, or 100% identical to that set forth in SEQ ID NO: 68.

In certain embodiments, the antibody that binds IGF1R comprises: (a)an immunoglobulin heavy chain CDR1 (CDR-H1) comprising the amino acid sequence set forth in SEQ ID NO: 71; (b) an immunoglobulin heavy chain CDR2 (CDR-H2) comprising the amino acid sequence set forth in SEQ ID NO: 72; (c) an immunoglobulin heavy chain CDR3 (CDR-H3) comprising the amino acid sequence set forth in SEQ ID NO: 73; (d) an immunoglobulin light chain CDR1 (CDR-L1) comprising the amino acid sequence set forth in SEQ ID NO: 74; (e) an immunoglobulin light chain CDR2 (CDR-L2) amino comprising the amino acid sequence set forth in SEQ ID NO: 75; and/or (f) and an immunoglobulin light chain CDR3 (CDR-L3) comprising the amino acid sequence set forth in SEQ ID NO: 76.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 77; and wherein the immunoglobulin light chain variable region comprises an amino acid sequence at least about 90%, 95%, 97%, 99%, or 100% identical to that set forth in SEQ ID NO: 78.

In certain embodiments, the antibody that binds IGF1R comprises: (a) an immunoglobulin heavy chain CDR1 (CDR-H1) comprising the amino acid sequence set forth in SEQ ID NO: 81; (b) an immunoglobulin heavy chain CDR2 (CDR-H2) comprising the amino acid sequence set forth in SEQ ID NO: 2; (c) an immunoglobulin heavy chain CDR3 (CDR-H3) comprising the amino acid sequence set forth in SEQ ID NO: 83; (d) an immunoglobulin light chain CDR1 (CDR-L1) comprising the amino acid sequence set forth in SEQ ID NO: 84; (e) an immunoglobulin light chain CDR2 (CDR-L2) amino comprising the amino acid sequence set forth in SEQ ID NO: 85; and/or (f) an immunoglobulin light chain CDR3 (CDR-L3) comprising the amino acid sequence set forth in SEQ ID NO: 86.

In certain embodiments, the antibody that binds IGF1R comprises an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 87; and wherein the immunoglobulin light chain variable region comprises an amino acid sequence at least about 90%, 95%, 97%, 99%, or 100% identical to that set forth in SEQ ID NO: 88.

In certain embodiments, the IGF1R inhibitory antibodies of this disclosure are administered at a therapeutically effective dosage and/or on a therapeutically acceptable schedule. In certain embodiments, therapeutically effective schedule is once weekly, once every two weeks, once every three weeks, once every four weeks, once every five weeks, once every six weeks, once every seven weeks, once every eight weeks, once every nine weeks, once every ten weeks, once every eleven weeks, or once every twelve weeks. In certain embodiments, the IGF1R inhibitory antibodies of this disclosure are administered from about 1 mg/kg per dose to about 50 mg/kg per dose. In certain embodiments, the IGF1R inhibitory antibodies of this disclosure are administered from about 5 mg/kg per dose to about 50 mg/kg per dose. In certain embodiments, the IGF1R inhibitory antibodies of this disclosure are administered from about 5 mg/kg per dose to about 40 mg/kg per dose. In certain embodiments, the IGF1R inhibitory antibodies of this disclosure are administered from about 5 mg/kg per dose to about 30 mg/kg per dose. In certain embodiments, the IGF1R inhibitory antibodies of this disclosure are administered from about 5 mg/kg per dose to about 25 mg/kg per dose. In certain embodiments, the IGF1R inhibitory antibodies of this disclosure are administered from about 5 mg/kg per dose to about 20 mg/kg per dose. In certain embodiments, the IGF1R inhibitory antibodies of this disclosure are administered from about 5 mg/kg per dose to about 15 mg/kg per dose. In certain embodiments, the IGF1R inhibitory antibodies of this disclosure are administered from about 5 mg/kg per dose to about 10 mg/kg per dose. In certain embodiments, the IGF1R inhibitory antibodies of this disclosure are administered from about 10 mg/kg per dose to about 30 mg/kg per dose. In certain embodiments, the IGF1R inhibitory antibodies of this disclosure are administered from about 10 mg/kg per dose to about 25 mg/kg per dose. In certain embodiments, the IGF1R inhibitory antibodies of this disclosure are administered from about 10 mg/kg per dose to about 20 mg/kg per dose. In certain embodiments, the IGF1R inhibitory antibodies of this disclosure are administered at about 1 mg/kg per dose. In certain embodiments, the IGF1R inhibitory antibodies of this disclosure are administered at about 2 mg/kg per dose. In certain embodiments, the IGF1R inhibitory antibodies of this disclosure are administered at about 3 mg/kg per dose. In certain embodiments, the IGF1R inhibitory antibodies of this disclosure are administered at about 4 mg/kg per dose. In certain embodiments, the IGF1R inhibitory antibodies of this disclosure are administered at about 5 mg/kg per dose. In certain embodiments, the IGF1R inhibitory antibodies of this disclosure are administered at about 10 mg/kg per dose. In certain embodiments, the IGF1R inhibitory antibodies of this disclosure are administered at about 15 mg/kg per dose. In certain embodiments, the IGF1R inhibitory antibodies of this disclosure are administered at about 20 mg/kg per dose. In certain embodiments, the IGF1R inhibitory antibodies of this disclosure are administered at about 25 mg/kg per dose. In certain embodiments, the IGF1R inhibitory antibodies of this disclosure are administered at about 30 mg/kg per dose. In certain embodiments, the IGF1R inhibitory antibodies of this disclosure are administered at about 35 mg/kg per dose. In certain embodiments, the IGF1R inhibitory antibodies of this disclosure are administered at about 40 mg/kg per dose. In certain embodiments, the IGF1R inhibitory antibodies of this disclosure are administered at about 45 mg/kg per dose. In certain embodiments, the IGF1R inhibitory antibodies of this disclosure are administered at about 50 mg/kg per dose.

In certain embodiments, the methods described herein comprise administering a first dose that is different than subsequent administered dose. In certain embodiments, the first dose is a loading dose that is higher than a subsequent maintenance dose. In certain embodiments, the first dose is a dose that is lower than a subsequent dose.

In certain embodiments, a first dose is administered from about 1 mg/kg to about 30 mg/kg; and a second dose is administer in a higher amount from about 1 mg/kg to about 30 mg/kg. In certain embodiments, a first dose is administered from about 5 mg/kg to about 30 mg/kg; and a second dose is administer in a higher amount from about 5 mg/kg to about 30 mg/kg. In certain embodiments, a first dose is administered from about 5 mg/kg to about 25 mg/kg; and a second dose is administer in a higher amount from about 5 mg/kg to about 25 mg/kg. In certain embodiments, a first dose is administered from about 10 mg/kg to about 20 mg/kg; and a second dose is administer in a higher amount from about 10 mg/kg to about 20 mg/kg. In certain embodiments, a first dose is administered from about 15 mg/kg to about 25 mg/kg; and a second dose is administer in a higher amount from about 15 mg/kg to about 25 mg/kg. In certain embodiments, a first dose is administered at about 10 mg/kg; and a second dose is administer at about 20 mg/kg.

In certain embodiments, the IGF1R inhibitory antibody for use with the methods of this disclosure is teprotumumab and is administered at a therapeutically effective dosage and/or on a therapeutically acceptable schedule. In certain embodiments, therapeutically effective schedule is once weekly, once every two weeks, once every three weeks, once every four weeks, once every five weeks, once every six weeks, once every seven weeks, once every eight weeks, once every nine weeks, once every ten weeks, once every eleven weeks, or once every twelve weeks. In certain embodiments, therapeutically effective schedule once every three weeks. In certain embodiments, therapeutically effective schedule once every four weeks. In certain embodiments, the IGF1R inhibitory antibodies of this disclosure are administered from about 1 mg/kg per dose to about 50 mg/kg per dose. In certain embodiments, teprotumumab is administered from about 5 mg/kg per dose to about 50 mg/kg per dose. In certain embodiments, teprotumumab is administered from about 5 mg/kg per dose to about 40 mg/kg per dose. In certain embodiments, teprotumumab is administered from about 5 mg/kg per dose to about 30 mg/kg per dose. In certain embodiments, teprotumumab is administered from about 5 mg/kg per dose to about 25 mg/kg per dose. In certain embodiments, teprotumumab is administered from about 5 mg/kg per dose to about 20 mg/kg per dose. In certain embodiments, teprotumumab is administered from about 5 mg/kg per dose to about 15 mg/kg per dose. In certain embodiments, teprotumumab is administered from about 5 mg/kg per dose to about 10 mg/kg per dose. In certain embodiments, teprotumumab is administered from about 10 mg/kg per dose to about 30 mg/kg per dose. In certain embodiments, teprotumumab is administered from about 10 mg/kg per dose to about 25 mg/kg per dose. In certain embodiments, teprotumumab is administered from about 10 mg/kg per dose to about 20 mg/kg per dose. In certain embodiments, teprotumumab is administered at about 1 mg/kg per dose. In certain embodiments, teprotumumab is administered at about 2 mg/kg per dose. In certain embodiments, teprotumumab is administered at about 3 mg/kg per dose. In certain embodiments, teprotumumab is administered at about 4 mg/kg per dose. In certain embodiments, teprotumumab is administered at about 5 mg/kg per dose. In certain embodiments, teprotumumab is administered at about 10 mg/kg per dose. In certain embodiments, teprotumumab is administered at about 15 mg/kg per dose. In certain embodiments, teprotumumab is administered at about 20 mg/kg per dose. In certain embodiments, teprotumumab is administered at about 25 mg/kg per dose. In certain embodiments, teprotumumab is administered at about 30 mg/kg per dose. In certain embodiments, teprotumumab is administered at about 35 mg/kg per dose. In certain embodiments, teprotumumab is administered at about 40 mg/kg per dose. In certain embodiments, teprotumumab is administered at about 45 mg/kg per dose. In certain embodiments, teprotumumab is administered at about 50 mg/kg per dose.

In certain embodiments, the methods described herein comprise administering a first dose of teprotumumab that is different than subsequent administered dose. In certain embodiments, the first dose of teprotumumab is a loading dose that is higher than a subsequent maintenance dose. In certain embodiments, the first dose is a dose that is lower than a subsequent dose.

In certain embodiments, a first dose of teprotumumab is administered from about 1 mg/kg to about 30 mg/kg; and a second dose of teprotumumab is administer in a higher amount from about 1 mg/kg to about 30 mg/kg. In certain embodiments, a first dose of teprotumumab is administered from about 5 mg/kg to about 30 mg/kg; and a second dose of teprotumumab is administer in a higher amount from about 5 mg/kg to about 30 mg/kg. In certain embodiments, a first dose of teprotumumab is administered from about 5 mg/kg to about 25 mg/kg; and a second dose of teprotumumab is administer in a higher amount from about 5 mg/kg to about 25 mg/kg. In certain embodiments, a first dose of teprotumumab is administered from about 10 mg/kg to about 20 mg/kg; and a second dose of teprotumumab is administer in a higher amount from about 10 mg/kg to about 20 mg/kg. In certain embodiments, a first dose of teprotumumab is administered from about 15 mg/kg to about 25 mg/kg; and a second dose of teprotumumab is administer in a higher amount from about 15 mg/kg to about 25 mg/kg. In certain embodiments, a first dose of teprotumumab is administered at about 10 mg/kg; and a second dose of teprotumumab is administer at about 20 mg/kg.

In certain embodiments, the IGF1R inhibitor comprises a non-antibody small molecule inhibitor of IGF1R signaling. In certain embodiments, the IGF1R inhibitor comprises picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof.

### Pharmaceutically Acceptable Excipients, Carriers, and Diluents

In certain embodiments the anti-IGF1R antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the IGF1R inhibitor antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits for treating inactive or chronic TED comprising one or more of the IGF1R inhibitors described herein in a suitable container and one or more additional components selected from: instructions for use; a diluent, an excipient, a carrier, and a device for administration. In certain embodiments the IGF1R inhibitor is an antibody of this disclosure. In certain embodiments, the antibody is teprotumumab.

In certain embodiments, described herein is a method of preparing a treatment for inactive or chronic TED comprising admixing one or more pharmaceutically acceptable excipients, carriers, or diluents and an IGF1R inhibitor of the current disclosure. In certain embodiments, described herein is a method of preparing a cancer treatment for storage or shipping comprising lyophilizing one or more antibodies of the current disclosure. In certain embodiments the IGF1R inhibitor is an antibody of this disclosure. In certain embodiments, the antibody is teprotumumab.

### EXAMPLES

The following illustrative examples are representative of embodiments of compositions and methods described herein and are not meant to be limiting in any way.

### Example 1—TEPEZZA® (teprotumumab-trbw) Treats Patients with Chronic (Inactive) Thyroid Eye Disease

The primary objective of the following example was to evaluate the efficacy, safety, and tolerability of TEPEZZA® (teprotumumab-trbw, hereinafter referred to as TEPEZZA) in comparison to placebo in treating patients with chronic (inactive) TED. In particular, measurements were taken to evaluate the effect of TEPEZZA versus placebo on the change of proptosis measurements in the study eye from Baseline at Week 24 in patients with chronic (inactive) TED. Measurements were also taken to determine proptosis responder rate (i.e., the percentage of patients with a≥2 mm reduction from Baseline without deterioration [≥2 mm increase] of proptosis in the fellow eye) at Week 24. The study also was used to evaluate the effect of TEPEZZA versus placebo on the change from Baseline at Week 24 in the Graves' Opthalmopathy Quality of Life (GO-QoL) questionnaire appearance and visual functioning subscales. The study also was used to evaluate the effect of TEPEZZA versus placebo on the change from Baseline at Week 24 in diplopia. The study also was used to evaluate the effect of TEPEZZA versus placebo on the binocular diplopia responder rate (i.e., the percentage of patients with Baseline diplopia>0 who have a reduction of≥1 grade). The study also was used to evaluate the effect of TEPEZZA versus placebo on the complete binocular diplopia responder rate (i.e., the percentage of patients with Baseline diplopia>0 and a score of 0 at Week 24).

### Trial Design

This was a randomized, double-masked, placebo-controlled, parallel-group, multicenter trial. Patients were screened for the trial within 4 weeks prior to the Baseline (Day 1) Visit. Approximately 57 patients who met the trial eligibility criteria were randomized on Day 1 in a 2:1 ratio to receive 8 infusions of TEPEZZA (10 mg/kg for the first infusion and 20 mg/kg for the remaining 7 infusions) or placebo once every 3 weeks (q3W). All patients entered a 24-week double-masked Treatment Period, during which trial drug was infused on Day 1 (Baseline) and Weeks 3, 6, 9, 12, 15, 18 and 21 (with a final visit at Week 24 of the 24-week Treatment Period).

All trial drug dosing was performed at a clinic or infusion center under adequate healthcare professional supervision. On each dosing day, scheduled assessments (except for Adverse Events (AEs) and concomitant medication use monitoring, which were monitored throughout the clinic visit) were completed prior to dosing. After each of the first 2 infusions, patients were contacted by phone/email the following day for safety and tolerability assessments. An additional phone/email contact and clinic visit was conducted for any patient experiencing an infusion-associated event.

At the end of the double-masked Treatment Period (Week 24), all patients were assessed for treatment response: proptosis responders (study eye has 2-mm reduction in proptosis from Baseline without deterioration [≥2-mm increase in proptosis] in fellow eye) or proptosis non-responders (study eye has<2-mm reduction in proptosis). Proptosis non-responders who have completed the double-masked Treatment Period may choose to receive 8 infusions of TEPEZZA (10 mg/kg for the first infusion and 20 mg/kg for the remaining 7 infusions) in an open-label fashion q3W at Weeks 24, 27, 30, 33, 36, 39, 42 and 45. These patients will return to the clinic at Week 48 for End-of-Treatment assessments, and patients will be contacted by phone/email 30 days after the Week 48 Visit for safety assessment. Proptosis responders, as well as non-responders who choose not to receive TEPEZZA in an open-label fashion, will enter a 30-day Follow-up Period, during which trial drug will not be administered. These patients will be contacted by phone/email 30 days after the Week 24 Visit for safety assessment.

Patients who prematurely discontinued trial drug dosing prior to Week 21 of the double-masked Treatment Period or prior to Week 45 of the open-label Treatment Period were asked to return to the clinic, undergo the scheduled End-of-Treatment assessments (with the exception of the collection of blood samples for biomarker evaluations) and enter the Follow-up Period, provided such continued participation will not detrimentally affect the health, safety and welfare of the patient per Investigator determination. An overview of the trial design is presented in Table 1.

TABLE 1
Trial Design Overview

| Screen | BL | Double-masked Treatment Period | | | | | | | Open-label Treatment Period [symbol] | | | | | | | | 30-day |
|---|---|---|---|---|---|---|---|---|---|---|---|---|---|---|---|---|---|
| | | TEPEZZA or Placebo [symbol] | | | | | | | (Proptosis Non-responders who Choose to Receive | | | | | | | | Follow- |
| | | 24 Weeks | | | | | | | Open-label TEPEZZA) | | | | | | | | up |
| | | | | | | | | | | | | | | | | | Period [symbol] |
| | * | * | * | * | * | * | * | * | * | * | * | * | * | * | * | * | * |
| 4 weeks | Day 1 [symbol] | W 3 [symbol] | W 6 | W 9 | W 12 | W 15 | W 18 | W 21 | W 24 [symbol] | W 27 [symbol] | W 30 | W 33 | W 36 | W 39 | W 42 | W 45 | W 48 [symbol] |
| predose | | | | | | | | | | | | | | | | | |

Randomization¹ <---------------------------------------------------------------------- Trial Week ---------------------------------------------------------------------->
* Infusion of trial drug (TEPEZZA or placebo in double-masked Treatment Period and open-label TEPEZZA in open-label Treatment Period).
BL = Baseline; q3W = once every 3 weeks; W = Week.

| | |
|---|---|
| 1. | Patients will be randomized in a 2:1 ratio to receive: |
| | a. TEPEZZA (10 mg/kg on Day 1 followed by 20 mg/kg q3W for the remaining 7 infusions); or |
| | b. Placebo (placebo q3W for all [symbol] infusions). |
| 2. | Visit windows are [symbol] 3 days for Weeks 3, 6, 9, 12, 15, 18 and 21 of double-masked Treatment Period and Weeks 27, 30, 33, |
| | 36, 39, 42 and 45 of open-label Treatment Period, and [symbol] 7 days for Week 24 of double-masked Treatment Period. Week 48 of |
| | open-label Treatment Period and 30-day Follow-up Period. |
| 3. | All patients will be contacted by phone/email the day after infusion for the first and second infusions during the double-masked |
| | Treatment Period and during the open-label Treatment Period, and thereafter as deemed appropriate; additional phone/email contacts |
| | will occur the day after any clinic visit where a patient experiences an infusion-related adverse event. |
| 4. | Patients who are proptosis non-responders at Week 24 of the double-masked Treatment Period will be offered the option to receive |
| | [symbol] infusions of TEPEZZA ( [symbol] mg/kg on Day [symbol] followed by 20 mg/kg for the remaining 7 infusions) in an open-label fashion. |
| 5. | All patients will be contacted via phone or email 30 days ( [symbol] 7 days) following the Week 24 (responders and non-responders |
| | who choose not to receive open-label TEPEZZA [symbol] or Week 48 (non-responders who receive open-label TEPEZZA) Visit. |

[symbol]

indicates data missing or illegible when filed

### Inclusion Criteria

Eligible patients were required to meet all of the following criteria.
1) Written informed consent.
2) Male or female at least 18 years old at Screening.
3) Initial diagnosis of TED≥2 years but<10 years prior to Screening. Clinical diagnosis of stable, chronic (inactive) TED, as determined by patient medical records indicating a Clinical Activity Score (CAS)≤1 in both eyes for at least 1 year prior to Screening or all of the following:
   a. no progression in proptosis for at least 1 year prior to Screening
   b. if patient has history of diplopia due to TED, no progression in diplopia for at least 1 year prior to Screening
   c. no new inflammatory TED symptoms for at least 1 year prior to Screening.
4) CAS≤1 at the Screening and Baseline Visits.
5) Proptosis≅3-mm increase from the patient's baseline (prior to diagnosis of TED), as estimated by treating physician and/or proptosis≥3 mm above normal for race and gender.
6) Patients must be euthyroid with the patient's baseline disease under control or have mild hypo- or hyperthyroidism (defined as free thyroxine and free triiodothyronine levels<50% above or below the normal limits) at Screening. Every effort should be made to correct the mild hypo- or hyperthyroidism promptly and to maintain the euthyroid state for the full duration of the trial.
7) Does not require immediate surgical ophthalmological intervention and is not planning corrective surgery/irradiation during the course of the trial.
8) Diabetic patients must have HbA1c≤8.0% at Screening.
9) Patients with a history of inflammatory bowel disease, ulcerative colitis or Crohn's disease must be in clinical remission for at least 3 months, with no history of bowel surgery within 6 months prior to screening and no planned surgery during the trial. Concomitant stable therapies for inflammatory bowel disease without modifications in the 3 months prior to Screening are allowed.
10) Women of childbearing potential (including those with an onset of menopause<2 years prior to Screening, non-therapy-induced amenorrhea for<12 months prior to Screening, or not surgically sterile [absence of ovaries and/or uterus]) must have a negative serum pregnancy test at Screening and negative urine pregnancy tests at all protocol-specified time points (i.e., prior to each dose and throughout patient's participation); patients who are sexually active with a non-vasectomized male partner must agree to use 2 reliable forms of contraception during the trial, 1 of which is recommended to be hormonal, such as an oral contraceptive. Hormonal contraception must be started at least 1 full cycle prior to Baseline and continue for 180 days after the last dose of trial drug. Highly effective contraceptive methods (failure rate<1% per year), when used consistently and correctly, include implants, injectables, combination oral contraceptives, some intrauterine devices, sexual abstinence and vasectomized partner.
11) Willing and able to comply with the prescribed treatment protocol and evaluations for the duration of the trial.

### Exclusion Criteria

Patients were ineligible for trial participation if they met any of the following criteria:
1) Decreased best-corrected visual acuity due to optic neuropathy, defined by a decrease in vision of 2 lines on the Snellen chart, new visual field defect or color defect secondary to optic nerve involvement within the last 6 months.
2) Corneal decompensation unresponsive to medical management in the study eye.
3) Decrease in proptosis of≥2 mm in the study eye between Screening and Baseline.
4) Prior orbital irradiation or orbital decompression in the study eye.
5) Prior strabismus surgery
6) Alanine aminotransferase or aspartate aminotransferase>3×the upper limit of normal or estimated glomerular filtration rate≤30 mL/min/1.73 m² at Screening.
7) Use of any steroid (intravenous, oral, steroid eye drops) for the treatment of TED or other conditions within 3 weeks prior to Screening. Steroids cannot be initiated during the trial. Exceptions include topical and inhaled steroids and steroids used to treat infusion reactions.
8) Any treatment with rituximab (Rituxan® or MabThera®) within 12 months prior to the first infusion of trial drug or tocilizumab (Actemra® or Roactemra®) within 6 months prior to the first infusion of trial drug. Use of any other non-steroid immunosuppressive agent within 3 months prior to the first infusion of trial drug.
9) Any previous treatment with TEPEZZA, including previous enrollment in this trial or participation in a prior teprotumumab trial.
10) Treatment with any monoclonal antibody within 3 months prior to Screening.
11) Identified pre-existing ophthalmic disease that, in the judgment of the Investigator, would preclude trial participation or complicate interpretation of trial results.
12) Use of an investigational agent for any condition within 60 days or 5 half-lives, whichever is longer, prior to Screening or anticipated use during the course of the trial.
13) Malignant condition in the past 12 months (except successfully treated basal/squamous cell carcinoma of the skin or cervical cancer in situ).
14) Pregnant or lactating women.
15) Current drug or alcohol abuse or history of either within the previous 2 years, in the opinion of the Investigator or as reported by the patient.
16) Known hypersensitivity to any of the components of TEPEZZA or prior hypersensitivity reactions to monoclonal antibodies.
17) Poorly controlled human immunodeficiency virus infection or untreated or positive viral load for hepatitis C or hepatitis B infections.
18) Any other condition that, in the opinion of the Investigator, would preclude inclusion in the trial.

### Patient Disposition

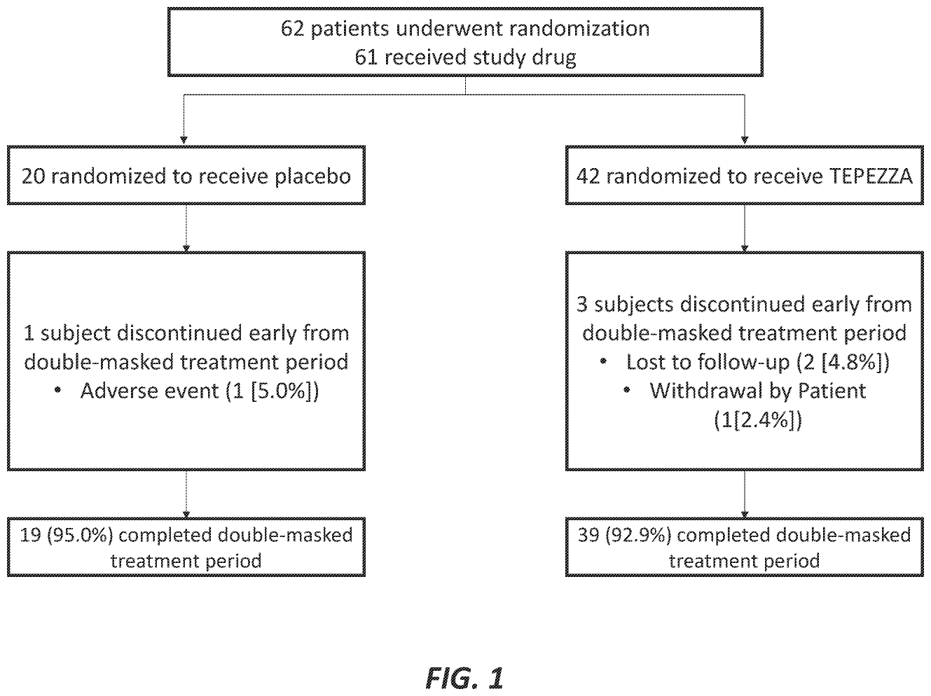

FIG. 1 illustrates patient disposition during the clinical trial. 20 patients received a placebo. Of the 20 patients that received the placebo, one patient (5%) discontinued early from the double-masked treatment period due to an adverse event. 19 patients (95%) that received the placebo completed the double-masked treatment period. 42 patients received TEPEZZA. Of the 42 patients that received the placebo, 3 patients discontinued early from the double-masked treatment period. Two patients (4.8%) were lost to follow-up, and one patient (2.4%) withdrew from the study. 39 patients (92.9%) that received TEPEZZA completed the double-masked treatment period. Table 2 summarizes the demographic and baseline characteristics of the patients enrolled in the study.

TABLE 2
Summary of Demographic and Baseline
Characteristics of Study Participants

| | Placebo | TEPEZZA |
|---|---|---|
| | (N = 20) | (N = 42) |
| Age (years) | | |
| Mean (SD) | 49.0 (16.45) | 48.6 (14.37) |
| Sex n (%) | | |
| Female | 18 (90.0) | 32 (76.2) |
| Male | 2 (10.0) | 10 (23.8) |
| Race n (%) | | |
| Asian | 1 (5.0) | 7 (16.7) |
| Black or African American | 5 (25.0) | 10 (23.8) |
| White | 12 (60.0) | 22 (52.4) |
| Other | 2 (10.0) | 3 (7.1) |
| Time Since Diagnosis of | | |
| Thyroid Eye Disease (years) | | |
| Mean (SD) | 5.4 (1.61) | 5.1 (1.88) |
| Median (Min-Max) | 5.8 (2.7-7.8) | 4.9 (2.2-8.7) |
| Tobacco Use n (%) | | |
| Former | 6 (30.0) | 11 (26.2) |
| Current | 2 (10.0) | 6 (14.3) |
| Never | 12 (60.0) | 25 (59.5) |
| Proptosis for Study Eye (mm) | | |
| Mean (SD) | 24.0 (2.82) | 24.6 (3.01) |
| Median (Min-Max) | 23.0 (20.0-28.0) | 25.0 (18.5, 31.0) |
| Proptosis for Fellow | | |
| Eye (mm) | | |
| Mean (SD) | 22.8 (3.23) | 23.5 (3.05) |
| Median (Min-Max) | 22.5 (17.0-28.0) | 24 (17.0-29.0) |
| Binocular Diplopia Score | | |
| n (%) | | |
| 0 | 16 (80.0) | 28 (66.7) |
| 1 | 2 (10.0) | 6 (14.3) |
| 2 | 1 (5.0) | 2 (4.8) |
| 3 | 1 (5.0) | 6 (14.3) |

As shown in Table 2, TEPEZZA has a numerically larger proptosis measurement at Baseline (median 25 mm vs. 23 mm). Additionally, TEPEZZA has numerically more diplopia at Baseline (33.3% vs. 20%).

### Dose Regimen/Route of Administration

All trial drug dosing was performed at a clinic or infusion center under adequate healthcare professional supervision. On Day 1 of the double-masked Treatment Period, patients were randomized in a 2:1 ratio to receive IV infusions of either: TEPEZZA (10 mg/kg on Day 1 followed by 20 mg/kg q3W for the remaining 7 infusions), or Placebo (q3W for all 8 infusions).

For non-responders that received open-label treatment following completion of the double-masked Treatment Period, all patients received 8 infusions of TEPEZZA (10 mg/kg for the first infusion followed by 20 mg/kg q3W for the remaining 7 infusions).

The first 2 infusions of the double-masked and open-label Treatment Periods were administered over approximately 90 minutes (but not less than 80 minutes). All subsequent infusions were administered over approximately 60 minutes (but not less than 50 minutes). For the first 3 infusions, patients were monitored for any AEs during the infusion and for 60 minutes after completion of the infusion. For subsequent infusions (if no previous infusion reactions), patients were monitored during and for 30 minutes after completion of the infusion. At any scheduled infusion, the infusion rate may have been reduced or the dose interrupted or held based on tolerability.

### Dosage Form and Formulation Strength

TEPEZZA 500 mg was provided in single-dose 20-mL glass vials as a freeze-dried powder. Each vial of TEPEZZA was reconstituted with 10 mL of sterile water for injection. The resulting solution had a concentration of 47.6 mg/mL teprotumumab antibody. Reconstituted TEPEZZA solution was further diluted in 0.9% (w/v) sodium chloride solution prior to administration.

Doses less than 1800 mg were administered in a total infusion volume of 100 mL, and doses 1800 mg and above were administered in a total infusion volume of 250 mL. To maintain a constant volume in the infusion bags, a volume equal to the volume of TEPEZZA to be placed into the infusion bag was first removed from the infusion bag using a sterile syringe and needle. The appropriate volume of reconstituted TEPEZZA solution based on the patient's dose and body weight was withdrawn from the reconstituted TEPEZZA vial(s) and transferred into an intravenous bag containing normal saline (0.9% sodium chloride) to prepare a diluted solution with total volume of 100 mL (for doses<1800 mg) or 250 mL (for doses≥1800 mg).

Placebo infusions consisted of a normal saline (0.9% NaCl) solution and was administered in 100 mL or 250 mL infusion bags, as appropriate, per weight-based dosing volumes.

### Duration of Treatment and Follow-Up

The planned of the double-masked Treatment Period was 24 weeks. The duration of the open-label Treatment Period was 24 weeks. Patients will enter a 30-day Follow-up Period at the end of either the double-masked or open-label Treatment Period.

### Criteria for Evaluation

At the Baseline (Day 1) Visit, the “study eye” (i.e., the eye with most significant proptosis) was identified. If both eyes are affected equally, the Investigator chose a “study eye.” Both eyes were assessed for efficacy, but the study eye was used to assess the primary efficacy endpoint.

Efficacy was assessed by proptosis (measured as proptosis evaluation of the Clinical Measures of Severity using a Hertel instrument provided by the Sponsor for consistency in measurement), binocular diplopia (measured as part of the Clinical Measures of Severity), orbital pain (using a 10-cm Visual Analog Scale (VAS)), quality of life (using the GO-QoL questionnaire) and, for patients at 1 investigative site, orbital MRI.

Safety was assessed via AE (including adverse events of special interest (AESI)) and concomitant medication use monitoring, immunogenicity testing, best-corrected visual acuity, vital signs, clinical safety laboratory evaluations (complete blood count and fasting chemistry [including thyroid panel and HbA1c]) and pregnancy testing (if applicable).

### Efficacy Endpoint Analysis

After Week 24, primary and secondary efficacy endpoints were met with statistical significance. Results are summarized in Table 3.

TABLE 3
Primary and First Secondary Efficacy Endpoints

| | Placebo | TEPEZZA | Difference |
|---|---|---|---|
| | (N = 20) | (N = 42) | (P-value) |
| Mean change from Baseline | −0.92 | −2.41 | −1.48 mm |
| at Week 24 in proptosis in | (0.323) | (0.228) | (0.0004) |
| the study eye, LS Mean (SE) | | | |
| Proptosis responder rate at | 5 | 26 | 36.9% |
| Week 24, n (%) | (25.0) | (61.9) | (0.0134) |
| Mean change from Baseline | VF: | VF: | 6.31 |
| at Week 24 in the GO-QoL | 2.41 | 8.73 | (0.0318) |
| questionnaire visual | (2.329) | (1.661) | 2.85 |
| functioning subscale (VF) | A: | A: | (0.6494) |
| and appearance subscale | 7.19 | 10.03 | |
| (A), LS Mean (SE) | (5.069) | (3.592) | |
| Change from Baseline at | | 2.13 | (0.3815) |
| Week 24 in diplopia as | | (0.39, 11.60) | |
| ordinal response categories, | | | |
| Odds Ratio (95% CI) | | | |
| Binocular diplopia | N = 4 | N = 14 | ~7.1% |
| responder rate at Week 24, n | 2 | 6 | (>0.9999) |
| (%) | (50.0) | (42.9) | |
| Complete binocular diplopia | N = 4 | N = 14 | 3.6% |
| responder rate | 1 | 4 | (>0.9999) |
| at week 24, n (%) | (25.0) | (28.6) | |

The primary efficacy analysis was conducted on the intent-to-treat (ITT) set. A Mixed-Model for Repeated-Measures (MMRM) analysis of covariance model fitting to the individual change from Baseline scores for the study eye was used for the analysis of change from Baseline in proptosis. The model included Baseline score, treatment group, visit, visit-by-treatment and visit-by-Baseline score as covariates. The unstructured covariance was used. The treatment difference (TEPEZZA minus placebo) based on the estimated least squares (LS) means at Week 24 will be presented with associated standard error (SE), 95% confidence interval (CI) and p-value.

The other endpoints of the proptosis responder rate, binocular diplopia responder rate and complete binocular diplopia responder rate were used to assess risk difference (difference in response rates). A 95% exact CI was provided for each risk difference observed between the treatment groups. Patients whose Week 24 evaluation was missing were considered treatment failures (non-responders) for the responder analysis. Change from Baseline at Week 24 in diplopia as ordinal response categories was analyzed by proportional odds model based on all randomized patients in the trial. If the assumption of proportional odds model did not hold, change from Baseline at Week 24 in diplopia will be analyzed using Fisher Exact test. Table 4 shows change from baseline at Week 24 in diplopia as ordinal response categories.

TABLE 4
Change from Baseline at Week 24 in Diplopia
as Ordinal Response Categories

| | Placebo | TEPEZZA |
|---|---|---|
| | (N = 20) | (N = 42) |
| Significant Improvement | 1 (5.0%) | 2 (4.8%) |
| Improvement | 1 (5.0%) | 4 (9.5%) |
| No change | 17 (85.0%) | 36 (85.7%) |
| Worsening | 1 (5.0%) | 0 |
| Significant Worsening | 0 | 0 |

Remaining other or exploratory endpoints analyzing change from Baseline (i.e., GQ-QoL, orbital pain by VAS, and MRI) used the same MMRM method specified for the primary efficacy endpoint. Descriptive summaries for observed and change from Baseline values in disease biomarkers were summarized by treatment group at each scheduled visit, which may be reported in CSR, standalone biomarker report or publication.

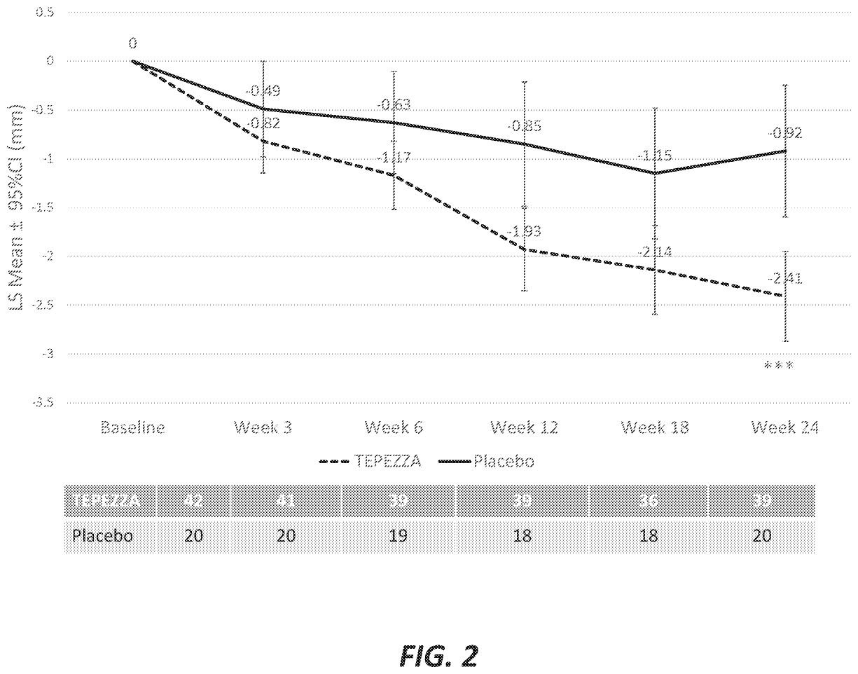

FIG. 2 illustrates the change in proptosis from baseline for patients with chronic (inactive) TED administered either teprotumumab (TEPEZZA®) or a placebo. As shown in FIG. 2, the mean change of proptosis (from baseline) at Week 24 for the TEPEZZA group was −2.41 mm. The mean change of proptosis (from baseline) at Week 24 for the placebo group was −0.92 mm.

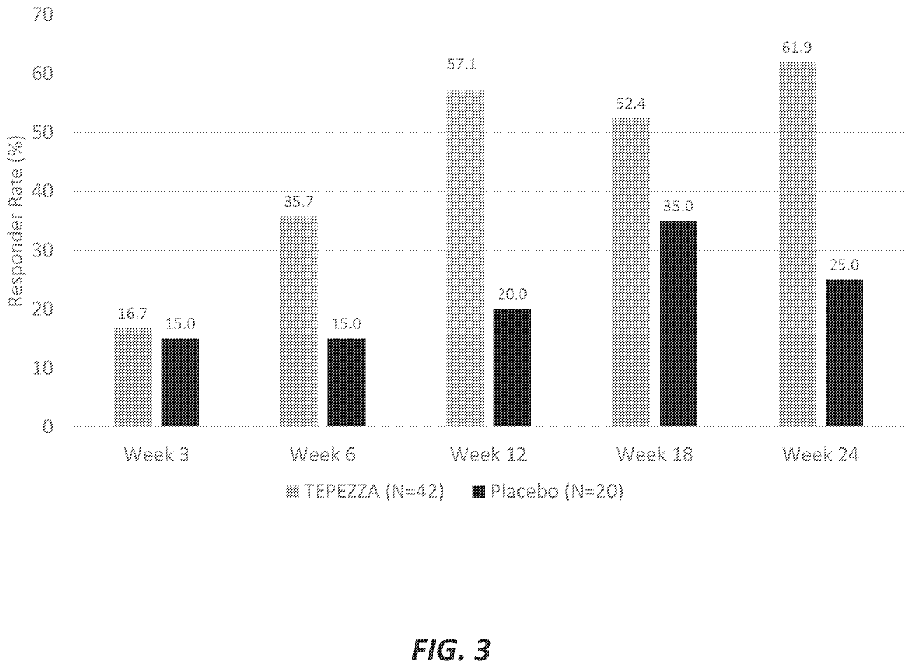

FIG. 3 illustrates proptosis responder rate for patients with chronic (inactive) TED administered either teprotumumab (TEPEZZA®) or a placebo. At Week 24, the TEPEZZA group had a 61.9% responder rate, compared to a 25.0% responder rate in the placebo group.

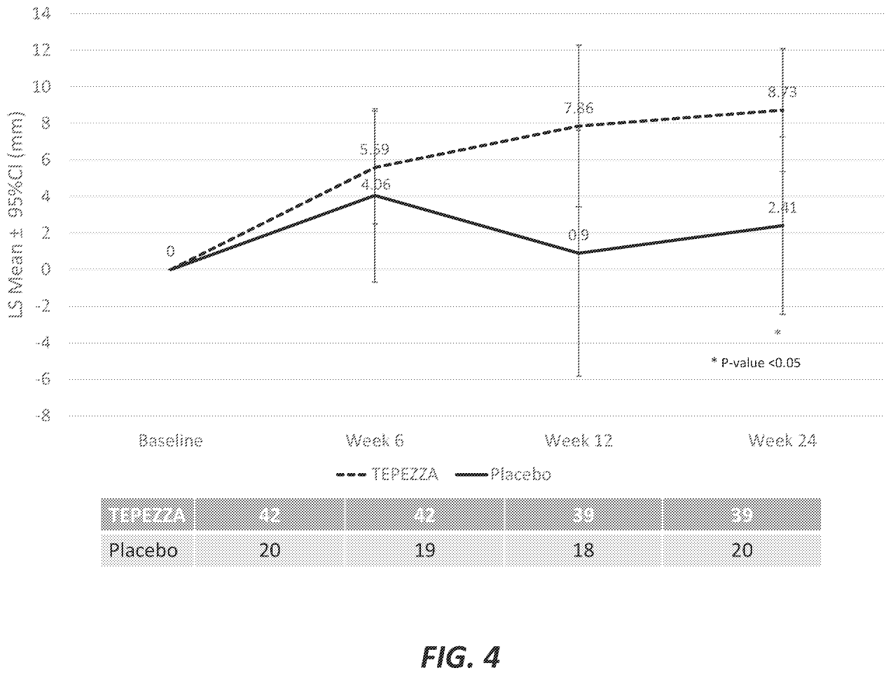

FIG. 4 illustrates the change in GO-QoL Visual Function from for patients with chronic (inactive) TED administered either teprotumumab (TEPEZZA®) or a placebo. As shown in FIG. 4, the mean change of GO-QoL Visual Function (from baseline) at Week 24 for the TEPEZZA group was 8.73. The mean change of GO-QoL Visual Function (from baseline) at Week 24 for the placebo group was 2.41 mm.

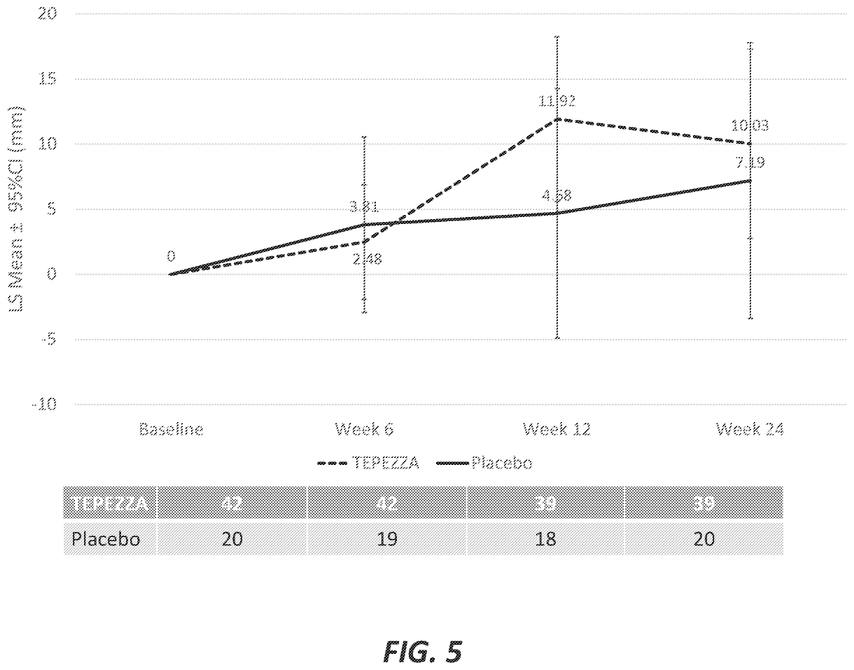

FIG. 5 illustrates the change in GO-QoL Appearance from baseline for patients with chronic (inactive) TED administered either teprotumumab (TEPEZZA®) or a placebo. As shown in FIG. 5, the mean change of GO-QoL Appearance (from baseline) at Week 24 for the TEPEZZA group was 10.03. The mean change of GO-QoL Appearance (from baseline) at Week 24 for the placebo group was 7.19.

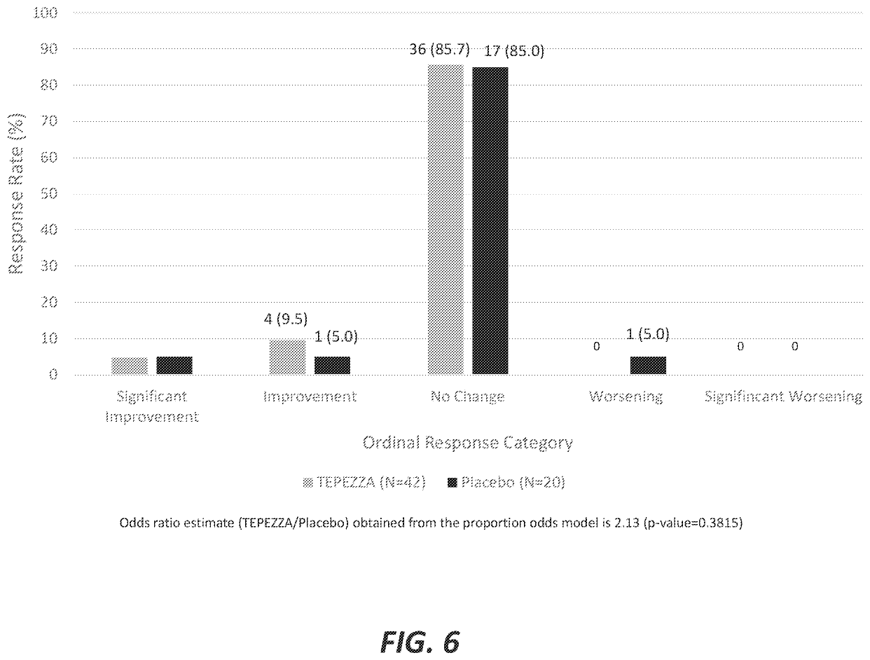

FIG. 6 illustrates the change in diplopia from baseline represented as response change for patients with chronic (inactive) TED administered either teprotumumab (TEPEZZA®) or a placebo.

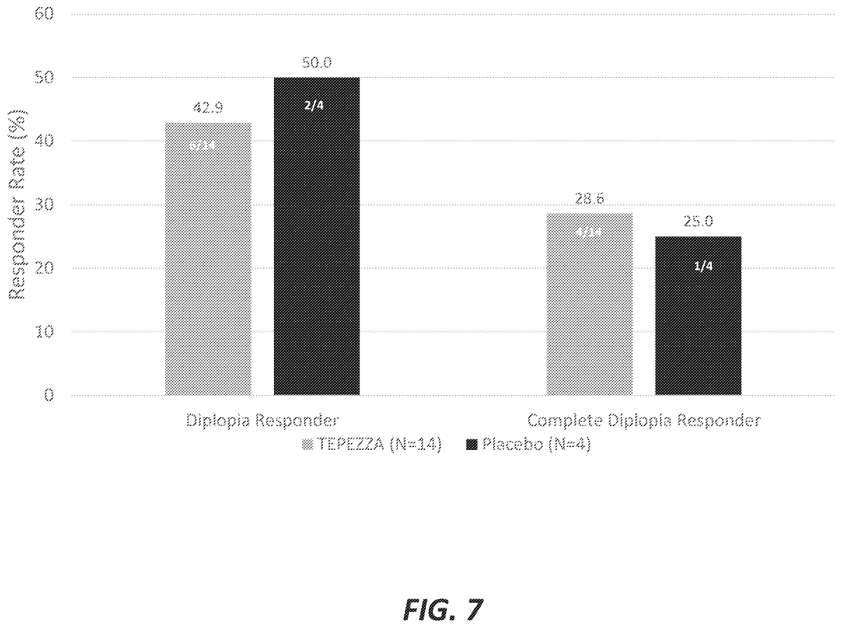

FIG. 7 illustrates diplopia responder rate and complete responder rates for patients with chronic (inactive) TED administered either teprotumumab (TEPEZZA®) or a placebo. As shown in FIG. 7, 6/14 patients in the TEPEZZA group were categorized as a diplopia responder, 4 of which were categorized as a complete diplopia responder (diplopia score of 0). For the placebo group, 2/4 patients were categorized as a diplopia responder, 1 of which was categorized as a complete diplopia responder (diplopia score of 0).

### Safety and Tolerability

The number and percentage of patients experiencing at least 1 occurrence of a treatment emergent adverse event (TEAE), serious adverse event (SAE), TEAE resulting in premature discontinuation of treatment, and adverse event of special interest (AESI) for each unique System Organ Class and Preferred Term was summarized by treatment group. TEAEs and SAEs were summarized by severity and relationship to trial drug, as assessed by the Investigator. Grade 3 and higher TEAEs, as graded by the National Cancer Institute Common Terminology Criteria for Adverse Events (NCI-CTCAE), were also summarized for each unique System Organ Class and Preferred Term.

The number and percentage of patients in each treatment group using concomitant medications were summarized by Anatomical Therapeutic Chemical Level 4 term and Preferred Term. For best-corrected visual acuity, shift tables were presented providing the count of patients in each treatment group with each type of finding (normal, abnormal—not clinically significant, or abnormal—clinically significant) at Baseline compared to each postbaseline visit. Descriptive summaries of observed and change from Baseline values were presented for each vital sign parameter by treatment group and visit. A shift table for vital signs by NCI-CTCAE grade and visit will be generated by treatment group.

Safety laboratory (hematology and fasting chemistry [including thyroid panel and HbA1c]) values and change from Baseline were summarized by visit and treatment group using descriptive statistics. The laboratory assessment were categorized as low, normal or high based on normal ranges and graded using the NCI-CTCAE grading scale, when available. Shift tables using categories of low, normal and high from Baseline to each visit were generated by treatment group. Additionally, a shift table for glucose by NCI-CTCAE grade and visit will be generated by treatment group. Summaries were provided separately for hyperglycemia.

The rate and titer of positive anti-drug antibody (ADA) samples were summarized by visit and treatment group using descriptive statistics. Teprotumumab peak and trough (i.e., prior to dose) concentrations were summarized by visit using descriptive statistics.

Records of adverse events are summarized in Tables 5, 6, and 7.

TABLE 5
Overall Summary of Treatment-Emergent Adverse Events

| During Double-masked Treatment Period | Placebo | TEPEZZA |
|---|---|---|
| n (%) | (N = 20) | (N = 41) |
| TEAEs | 16 (80.0) | 33 (80.5) |
| Treatment-Related TEAEs | 12 (60.0) | 31 (75.6) |
| CTCAE Criteria Grade 3 or Higher TEAEs | 1 (5.0) | 1 (2.4) |
| Serious TEBAEs | 1 (5.0) | 1 (2.4) |
| Treatment-Related Serious TEAEs | 1 (5.0) | 1 (2.4) |
| TEAEs Resulting in Study Drug | 1 (5.0) | 1 (2.4) * |
| Discontinuation | | |
| TEAEs of Special Interest | 7 (35.0) | 15 (36.6) |
| TEAEs Leading to Death | 0 | 0 |

TABLE 6
TEAEs of Special Interest

| During Double-masked Treatment Period | Placebo | TEPEZZA |
|---|---|---|
| n (%) | (N = 20) | (N = 41) |
| Any TEAE of Special Interest | 7 (35.0) | 15 (36.6) |
| Infusion Reactions | 3 (15.0) | 2 (4.9) |
| Hyperglycemia | 2 (10.0) | 6 (14.6) |
| Hearing Impairment | 2 (10.0) | 9 (22.0) |
| New onset Inflammatory Bowel Disease and | 0 | 0 |
| Exacerbation of Inflammatory Bowel Disease | | |

TABLE 7
Occurrence of Hearing Impairment Events

| During Double-masked | Placebo (N = 20) | | TEPEZZA (N = 41) | |
|---|---|---|---|---|
| Treatment Period n (%) | Patients | Events | Patients | Events |
| Hearing Impairment | 2 (10.0) | 3 | 9 (22.0) | 12 |
| Autophony | — | | 1 (2.4) | 1 |
| Conductive deafness | — | | 1 (2.4) | 1 |
| Deafness unilateral | — | | 1 (2.4) | 2 |
| Eustachian tube dysfunction | — | | 1 (2.4) | 1 |
| Hypoacusis | — | | 4 (9.8) | 4 |
| Tinnitus | 2 (10.0) | 3 | 2 (4.9) | 2 |
| Tympanic membrane disorder | — | | 1 (2.4) | 1 |

### Embodiments

Provided herein are methods and compositions for the treatment of thyroid eye disease and related conditions, as illustrated by the following embodiments.

Embodiment 1. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 6 months or longer.

Embodiment 2. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 7 months or longer.

Embodiment 3. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 8 months or longer.

Embodiment 4. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 9 months or longer.

Embodiment 5. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 10 months or longer.

Embodiment 6. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 11 months or longer.

Embodiment 7. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 12 months or longer.

Embodiment 8. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 13 months or longer.

Embodiment 9. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 14 months or longer.

Embodiment 10. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 15 months or longer.

Embodiment 11. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 16 months or longer.

Embodiment 12. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 17 months or longer.

Embodiment 13. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 18 months or longer.

Embodiment 14. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 19 months or longer.

Embodiment 15. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 20 months or longer.

Embodiment 16. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 21 months or longer.

Embodiment 17. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 22 months or longer.

Embodiment 18. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 23 months or longer.

Embodiment 19. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 24 months or longer.

Embodiment 20. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 25 months or longer.

Embodiment 21. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 26 months or longer.

Embodiment 22. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 27 months or longer.

Embodiment 23. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 28 months or longer.

Embodiment 24. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 29 months or longer.

Embodiment 25. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 30 months or longer.

Embodiment 26. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 31 months or longer.

Embodiment 27. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 32 months or longer.

Embodiment 28. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 33 months or longer.

Embodiment 29. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 34 months or longer.

Embodiment 30. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 35 months or longer.

Embodiment 31. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 2 for 36 months or longer.

Embodiment 32. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 6 months or longer.

Embodiment 33. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 7 months or longer.

Embodiment 34. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 8 months or longer.

Embodiment 35. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 9 months or longer.

Embodiment 36. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 10 months or longer.

Embodiment 37. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 11 months or longer.

Embodiment 38. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 12 months or longer.

Embodiment 39. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 13 months or longer.

Embodiment 40. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 14 months or longer.

Embodiment 41. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 15 months or longer.

Embodiment 42. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 16 months or longer.

Embodiment 43. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 17 months or longer.

Embodiment 44. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 18 months or longer.

Embodiment 45. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 19 months or longer.

Embodiment 46. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 20 months or longer.

Embodiment 47. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 21 months or longer.

Embodiment 48. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 22 months or longer.

Embodiment 49. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 23 months or longer.

Embodiment 50. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 24 months or longer.

Embodiment 51. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 25 months or longer.

Embodiment 52. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 26 months or longer.

Embodiment 53. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 27 months or longer.

Embodiment 54. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 28 months or longer.

Embodiment 55. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 29 months or longer.

Embodiment 56. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 30 months or longer.

Embodiment 57. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 31 months or longer.

Embodiment 58. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 32 months or longer.

Embodiment 59. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 33 months or longer.

Embodiment 60. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 34 months or longer.

Embodiment 61. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 35 months or longer.

Embodiment 62. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of less than 1 for 36 months or longer.

Embodiment 63. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 6 months or longer.

Embodiment 64. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 7 months or longer.

Embodiment 65. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 8 months or longer.

Embodiment 66. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 9 months or longer.

Embodiment 67. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 10 months or longer.

Embodiment 68. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 11 months or longer.

Embodiment 69. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 12 months or longer.

Embodiment 70. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 13 months or longer.

Embodiment 71. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 14 months or longer.

Embodiment 72. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 15 months or longer.

Embodiment 73. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 16 months or longer.

Embodiment 74. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 17 months or longer.

Embodiment 75. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 18 months or longer.

Embodiment 76. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 19 months or longer.

Embodiment 77. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 20 months or longer.

Embodiment 78. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 21 months or longer.

Embodiment 79. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 22 months or longer.

Embodiment 80. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 23 months or longer.

Embodiment 81. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 24 months or longer.

Embodiment 82. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 25 months or longer.

Embodiment 83. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 26 months or longer.

Embodiment 84. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 27 months or longer.

Embodiment 85. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 28 months or longer.

Embodiment 86. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 29 months or longer.

Embodiment 87. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 30 months or longer.

Embodiment 88. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 31 months or longer.

Embodiment 89. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 32 months or longer.

Embodiment 90. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 33 months or longer.

Embodiment 91. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 34 months or longer.

Embodiment 92. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 35 months or longer.

Embodiment 93. A method of treating an individual with thyroid eye disease (TED), the method comprising administering an insulin-like growth factor 1 receptor (IGF1R) inhibitor to the individual with TED, wherein the individual with TED has a Clinical Activity Score (CAS) of 0 for 36 months or longer.

Embodiment 94. A method of treating an individual with inactive or chronic thyroid eye disease (TED), the method comprising administering to an individual with inactive or chronic TED an insulin-like growth factor 1 receptor (IGF1R) inhibitor, thereby treating the inactive or chronic TED, optionally wherein the IGF1R inhibitor is not teprotumumab.

Embodiment 95. The method of Embodiment 94, wherein the inactive or chronic TED is associated with Grave's disease.

Embodiment 96. The method of Embodiment 94 or 95, wherein the inactive or chronic TED has a Clinical Activity Score (CAS) of 1 or less.

Embodiment 97. The method of any one of Embodiments 94 to 96, wherein the inactive or chronic TED has a Clinical Activity Score (CAS) of 0.

Embodiment 98. The method of any one of Embodiments 94 to 97, wherein the inactive TED is chronic TED.

Embodiment 99. The method of Embodiment 98, wherein the chronic TED has been inactive for at least 6 months prior to treatment.

Embodiment 100. The method of any one of Embodiments 94 to 97, wherein the inactive or chronic TED has been inactive for at least 12 months prior to treatment.

Embodiment 101. The method of any one of Embodiments 94 to 97, wherein the inactive or chronic TED has been inactive for at least 24 months prior to treatment.

Embodiment 102. The method of any one of Embodiments 94 to 101, wherein the inactive or chronic TED has been previously treated with an IGF1R inhibitor.

Embodiment 103. The method of Embodiment 102, wherein the IGF1R inhibitor comprises ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof.

Embodiment 104. The method of any one of Embodiments 94 to 103, wherein the IGF1R inhibitor comprises an antibody that binds IGF1R.

Embodiment 105. The method of any one of Embodiments 94 to 104, wherein the antibody that binds IGF1R is chimeric or humanized.

Embodiment 106. The method of any one of Embodiments 94 to 105, wherein the antibody that binds IGF1R is an IgG antibody.

Embodiment 107. The method of any one of Embodiments 94 to 106, wherein the antibody that binds IGF1R is a Fab, F(ab)₂, a single-domain antibody, or a single chain variable fragment (scFv).

Embodiment 108. The method of any one of Embodiments 94 to 107, wherein the IGF1R inhibitor is an antibody comprising ganitumab, figitumumab, MEDI-573, cixutumumab, dalotuzumab, robatumumab, robatumumab, AVE1642, BIIB022, xentuzumab, istiratumab, or any combination thereof.

Embodiment 109. The method of any one of Embodiments 94 to 108, wherein the IGF1R inhibitor is an antibody comprising:
a. an immunoglobulin heavy chain CDR1 (CDR-H1) comprising the amino acid sequence set forth in any one of SEQ ID NO: 11, 21, 31, 41, 51, 61, 71, or 81;
b. an immunoglobulin heavy chain CDR2 (CDR-H2) comprising the amino acid sequence set forth in any one of SEQ ID NO: 12, 22, 32, 42, 52, 62, 72, or 82;
c. an immunoglobulin heavy chain CDR3 (CDR-H3) comprising the amino acid sequence set forth in any one of SEQ ID NO: 13, 23, 33, 43, 53, 63, 73, or 83;
d. an immunoglobulin light chain CDR1 (CDR-L1) comprising the amino acid sequence set forth in any one of SEQ ID NO: 14, 24, 34, 44, 54, 64, 74, or 84;
e. an immunoglobulin light chain CDR2 (CDR-L2) amino comprising the amino acid sequence set forth in any one of SEQ ID NO: 15, 25, 35, 45, 55, 65, 75, or 85; and/or
f. and an immunoglobulin light chain CDR3 (CDR-L3) comprising the amino acid sequence set forth in any one of SEQ ID NO: 16, 26, 36, 46, 56, 66, 76, or 86.

Embodiment 110. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 11;
b. an immunoglobulin heavy chain CDR2 (CDR-H2) comprising the amino acid sequence set forth in SEQ ID NO: 12;
c. an immunoglobulin heavy chain CDR3 (CDR-H3) comprising the amino acid sequence set forth in SEQ ID NO: 13;
d. an immunoglobulin light chain CDR1 (CDR-L1) comprising the amino acid sequence set forth in SEQ ID NO: 14;
e. an immunoglobulin light chain CDR2 (CDR-L2) amino comprising the amino acid sequence set forth in SEQ ID NO: 15; and/or
f. and an immunoglobulin light chain CDR3 (CDR-L3) comprising the amino acid sequence set forth in SEQ ID NO: 16.

Embodiment 111. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 21;
b. an immunoglobulin heavy chain CDR2 (CDR-H2) comprising the amino acid sequence set forth in SEQ ID NO: 22;
c. an immunoglobulin heavy chain CDR3 (CDR-H3) comprising the amino acid sequence set forth in SEQ ID NO: 23;
d. an immunoglobulin light chain CDR1 (CDR-L1) comprising the amino acid sequence set forth in SEQ ID NO: 24;
e. an immunoglobulin light chain CDR2 (CDR-L2) amino comprising the amino acid sequence set forth in SEQ ID NO: 25; and/or
f. and an immunoglobulin light chain CDR3 (CDR-L3) comprising the amino acid sequence set forth in SEQ ID NO: 26.

Embodiment 112. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 31;
b. an immunoglobulin heavy chain CDR2 (CDR-H2) comprising the amino acid sequence set forth in SEQ ID NO: 32;
c. an immunoglobulin heavy chain CDR3 (CDR-H3) comprising the amino acid sequence set forth in SEQ ID NO: 33;
d. an immunoglobulin light chain CDR1 (CDR-L1) comprising the amino acid sequence set forth in SEQ ID NO: 34;
e. an immunoglobulin light chain CDR2 (CDR-L2) amino comprising the amino acid sequence set forth in SEQ ID NO: 35; and/or
f. and an immunoglobulin light chain CDR3 (CDR-L3) comprising the amino acid sequence set forth in SEQ ID NO: 36.

Embodiment 113. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 41;
b. an immunoglobulin heavy chain CDR2 (CDR-H2) comprising the amino acid sequence set forth in SEQ ID NO: 42;
c. an immunoglobulin heavy chain CDR3 (CDR-H3) comprising the amino acid sequence set forth in SEQ ID NO: 43;
d. an immunoglobulin light chain CDR1 (CDR-L1) comprising the amino acid sequence set forth in SEQ ID NO: 44;
e. an immunoglobulin light chain CDR2 (CDR-L2) amino comprising the amino acid sequence set forth in SEQ ID NO: 45; and/or
f. and an immunoglobulin light chain CDR3 (CDR-L3) comprising the amino acid sequence set forth in SEQ ID NO: 46.

Embodiment 114. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 51;
b. an immunoglobulin heavy chain CDR2 (CDR-H2) comprising the amino acid sequence set forth in SEQ ID NO: 52;
c. an immunoglobulin heavy chain CDR3 (CDR-H3) comprising the amino acid sequence set forth in SEQ ID NO: 53;
d. an immunoglobulin light chain CDR1 (CDR-L1) comprising the amino acid sequence set forth in SEQ ID NO: 54;
e. an immunoglobulin light chain CDR2 (CDR-L2) amino comprising the amino acid sequence set forth in SEQ ID NO: 55; and/or
f. and an immunoglobulin light chain CDR3 (CDR-L3) comprising the amino acid sequence set forth in SEQ ID NO: 56.

Embodiment 115. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 61;
b. an immunoglobulin heavy chain CDR2 (CDR-H2) comprising the amino acid sequence set forth in SEQ ID NO: 62;
c. an immunoglobulin heavy chain CDR3 (CDR-H3) comprising the amino acid sequence set forth in SEQ ID NO: 63;
d. an immunoglobulin light chain CDR1 (CDR-L1) comprising the amino acid sequence set forth in SEQ ID NO: 64;
e. an immunoglobulin light chain CDR2 (CDR-L2) amino comprising the amino acid sequence set forth in SEQ ID NO: 65; and/or
f. and an immunoglobulin light chain CDR3 (CDR-L3) comprising the amino acid sequence set forth in SEQ ID NO: 66.

Embodiment 116. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 71;
b. an immunoglobulin heavy chain CDR2 (CDR-H2) comprising the amino acid sequence set forth in SEQ ID NO: 72;
c. an immunoglobulin heavy chain CDR3 (CDR-H3) comprising the amino acid sequence set forth in SEQ ID NO: 73;
d. an immunoglobulin light chain CDR1 (CDR-L1) comprising the amino acid sequence set forth in SEQ ID NO: 74;
e. an immunoglobulin light chain CDR2 (CDR-L2) amino comprising the amino acid sequence set forth in SEQ ID NO: 75; and/or
f. and an immunoglobulin light chain CDR3 (CDR-L3) comprising the amino acid sequence set forth in SEQ ID NO: 76.

Embodiment 117. The method of any one of Embodiments 94 to 108, wherein the IGF1R inhibitor is an antibody comprising:
a. an immunoglobulin heavy chain CDR1 (CDR-H1) comprising the amino acid sequence set forth in SEQ ID NO: 81;
b. an immunoglobulin heavy chain CDR2 (CDR-H2) comprising the amino acid sequence set forth in SEQ ID NO: 82;
c. an immunoglobulin heavy chain CDR3 (CDR-H3) comprising the amino acid sequence set forth in SEQ ID NO: 83;
d. an immunoglobulin light chain CDR1 (CDR-L1) comprising the amino acid sequence set forth in SEQ ID NO: 84;
e. an immunoglobulin light chain CDR2 (CDR-L2) amino comprising the amino acid sequence set forth in SEQ ID NO: 85; and/or
f. and an immunoglobulin light chain CDR3 (CDR-L3) comprising the amino acid sequence set forth in SEQ ID NO: 86.

Embodiment 118. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18.

Embodiment 119. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 27; and wherein the immunoglobulin light chain variable region comprises an amino acid sequence at least about 90%, 95%, 97%, 99%, or 100% identical to that set forth in SEQ ID NO: 28.

Embodiment 120. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 37; and wherein the immunoglobulin light chain variable region comprises an amino acid sequence at least about 90%, 95%, 97%, 99%, or 100% identical to that set forth in SEQ ID NO: 38.

Embodiment 121. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 47; and wherein the immunoglobulin light chain variable region comprises an amino acid sequence at least about 90%, 95%, 97%, 99%, or 100% identical to that set forth in SEQ ID NO: 48.

Embodiment 122. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 57; and wherein the immunoglobulin light chain variable region comprises an amino acid sequence at least about 90%, 95%, 97%, 99%, or 100% identical to that set forth in SEQ ID NO: 58.

Embodiment 123. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 67; and wherein the immunoglobulin light chain variable region comprises an amino acid sequence at least about 90%, 95%, 97%, 99%, or 100% identical to that set forth in SEQ ID NO: 68.

Embodiment 124. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 77; and wherein the immunoglobulin light chain variable region comprises an amino acid sequence at least about 90%, 95%, 97%, 99%, or 100% identical to that set forth in SEQ ID NO: 78.

Embodiment 125. The method of any one of Embodiments 94 to 108, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 87; and wherein the immunoglobulin light chain variable region comprises an amino acid sequence at least about 90%, 95%, 97%, 99%, or 100% identical to that set forth in SEQ ID NO: 88.

Embodiment 126. The method of any one of Embodiments 94 to 108, wherein the IGF1R inhibitor is an antibody or antigen binding fragment thereof comprising an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 9; and wherein the immunoglobulin light chain comprises an amino acid sequence at least about 90%, 95%, 97%, 99%, or 100% identical to that set forth in SEQ ID NO: 10.

Embodiment 127. The method of any one of Embodiments 94 to 126, wherein the IGF1R inhibitor inhibits IGF1R signaling.

Embodiment 128. The method of any one of Embodiments 94 to 127, wherein the IGF1R inhibitor is administered at a dose of about 5 mg/kg to about 50 mg/kg.

Embodiment 129. The method of any one of Embodiments 94 to 128, wherein the IGF1R inhibitor is administered at a first dose of about 10 mg/kg and at a subsequent dose of about 20 mg/kg.

Embodiment 130. The method of any one of Embodiments 94 to 129, wherein the IGF1R inhibitor is administered once every three weeks.

Embodiment 131. The method of any one of Embodiments 94 to 130, wherein the IGF1R inhibitor is included in a pharmaceutical formulation comprising a pharmaceutically acceptable excipient, carrier, or diluent.

Embodiment 132. The method of Embodiment 131, wherein the pharmaceutical formulation is formulated for intravenous administration.

Embodiment 133. The method of Embodiment 131, wherein the pharmaceutical formulation is formulated for subcutaneous administration.

Embodiment 134. The method of any one of Embodiments 94 to 133, wherein the IGF1R inhibitor reduces proptosis in the individual with inactive or chronic TED.

Embodiment 135. The method of Embodiment 134, wherein the IGF1R inhibitor reduces proptosis by at least about 2 mm in the individual with inactive or chronic TED.

Embodiment 136. The method of Embodiment 134, wherein the IGF1R inhibitor reduces proptosis by at least about 3 mm in the individual with inactive or chronic TED.

Embodiment 137. The method of any one of Embodiments 94 to 136, wherein the IGF1R inhibitor reduces diplopia in the individual with inactive or chronic TED.

Embodiment 138. The method of Embodiment 137, wherein the IGF1R inhibitor reduces a diplopia score in the individual with inactive or chronic TED by at least 2 grades.

Embodiment 139. The method of Embodiment 137, wherein the IGF1R inhibitor reduces a diplopia score in the individual with inactive or chronic TED to 0.

Embodiment 140. The method of any one of Embodiments 94 to 139, wherein the IGF1R inhibitor reduces binocular diplopia in the individual with inactive or chronic TED.

Embodiment 141. The method of Embodiment 140, wherein the IGF1R inhibitor reduces a binocular diplopia score in the individual with inactive or chronic TED by at least 2 grades.

Embodiment 142. The method of Embodiment 140, wherein the IGF1R inhibitor reduces a binocular diplopia score in the individual with inactive or chronic TED to 0.

Embodiment 143. The method of any one of Embodiments 94 to 142 wherein the individual with the inactive or chronic TED has not been previously treated with an IGF1R inhibitor.

Embodiment 144. The method of any one of Embodiments 94 to 143, wherein the individual with the inactive or chronic TED has not previously undergone orbital irradiation.

Embodiment 145. The method of any one of Embodiments 94 to 144, wherein the individual with the inactive or chronic TED has not previously undergone orbital decompression surgery.

Embodiment 146. The method of any one of Embodiments 94 to 145, wherein the individual with the inactive or chronic TED has not previously undergone strabismus surgery.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

TABLE 8
Sequences Described Herein

| SEQ ID | | |
|---|---|---|
| NO: | Sequence | Origin |
| 1 | SYGMH | |
| 2 | IIWFDGSSTYYADSVRG | |
| 3 | ELGRRYFDL | |
| 4 | RASQSVSSYLA | |
| 5 | DASKRAT | |
| 6 | QQRSKWPPWTF | |
| 7 | QVELVESGGGVVQPGRSQRLSCAASGFTFSSYGMHWVRQAPGK | |
| | GLEWVAIIWFDGSSTYYADSVRGRFTISRDNSKNTLYLQMNSLR | |
| | AEDTAVYFCARELGRRYFDLWGRGTLVSVSS | |
| 8 | EIVLTQSPATLSLSPGERATLSCRASQSVSSYLAWYQQKPGQAPR | |
| | LLIYDASKRATGIPARFSGSGSGTDFTLTISSLEPEDFAVYYCQQR | |
| | SKWPPWTFGQGTKVESK | |
| 9 | QVELVESGGGVVQPGRSQRLSCAASGFTFSSYGMHWVRQAPGK | |
| | GLEWVAIIWFDGSSTYYADSVRGRFTISRDNSKNTLYLQMNSLR | |
| | AEDTAVYFCARELGRRYFDLWGRGTLVSVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPGK | |
| 10 | EIVLTQSPATLSLSPGERATLSCRASQSVSSYLAWYQQKPGQAPR | |
| | LLIYDASKRATGIPARFSGSGSGTDFTLTISSLEPEDFAVYYCQQR | |
| | SKWPPWTFGQGTKVESKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| 11 | GYTFTSYW | |
| 12 | INPSNGRT | |
| 13 | ARGRPDYYGSSKWYFDV | |
| 14 | QSIVHSNVNTY | |
| 15 | KVS | |
| 16 | FQGSHVPPT | |
| 17 | QVQLVQSGAEVVKPGASVKLSCKASGYTFTSYWMHWVKQRPG | |
| | QGLEWIGEINPSNGRTNYNQKFQGKATLTVDKSSSTAYMQLSSL | |
| | TSEDSAVYYFARGRPDYYGSSKWYFDVWGQGTTVTVSS | |
| 18 | DVVMTQTPLSLPVSLGDPASISCRSSQSIVHSNVNTYLEWYLQKP | |
| | GQSPRLLIYKVSNRFSGVPDRFSGSGAGTDFTLRISRVEAEDLGIY | |
| | YCFQGSHVPPTFGGGTKLEIKR | |
| 19 | | |
| 20 | | |
| 21 | GYSITGGYL | |
| 22 | ISYDGTN | |
| 23 | ARYGRVFFDY | |
| 24 | QSIVHSNGNTY | |
| 25 | KVS | |
| 26 | FQGSHVPWT | |
| 27 | QVQLQESGPGLVKPSETLSLTCTVSGYSITGGYLWNWIRQPPGK | |
| | GLEWIGYISYDGTNNYKPSLKDRVTISRDTSKNQFSLKLSSVTAA | |
| | DTAVYYCARYGRVFFDYWGQGTLVTVSS | |
| 28 | DIVMTQSPLSLPVTPGEPASISCRSSQSIVHSNGNTYLQWYLQKP | |
| | GQSPQLLIYKVSNRLYGVPDRFSGSGSGTDFTLKISRVEAEDVGV | |
| | YYCFQGSHVPWTFGQGTKVEIKR | |
| 29 | | |
| 30 | | |
| 31 | GGTFSSYA | |
| 32 | IIPIFGTA | |
| 33 | ARAPLRFLEWSTQDHYYYYYMDV | |
| 34 | SLRSYY | |
| 35 | GEN | |
| 36 | KSRDGSGQHLV | |
| 37 | EVQLVQSGAEVKKPGSSVKVSCKASGGTFSSYAISWVRQAPGQ | |
| | GLEWMGGIIPIFGTANYAQKFQGRVTITADKSTSTAYMELSSLRS | |
| | EDTAVYYCARAPLRFLEWSTQDHYYYYYMDVWGKGTTVTVSS | |
| 38 | SSELTQDPAVSVALGQTVRITCQGDSLRSYYATWYQQKPGQAPI | |
| | LVIYGENKRPSGIPDRFSGSSSGNTASLTITGAQAEDEADYYCKS | |
| | RDGSGQHLVFGGGTKLTVLGQPK | |
| 39 | | |
| 40 | | |
| 41 | GFTFSSYA | |
| 42 | ISGSGGTT | |
| 43 | AKDLGWSDSYYYYYGMDV | |
| 44 | QGIRND | |
| 45 | AAS | |
| 46 | LQHNSYPCS | |
| 47 | EVQLLESGGGLVQPGGSLRLSCTASGFTFSSYAMNWVRQAPGK | |
| | GLEWVSAISGSGGTTFYADSVKGRFTISRDNSRTTLYLQMNSLR | |
| | AEDTAVYYCAKDLGWSDSYYYYYGMDVWGQGTTVTVSS | |
| 48 | DIQMTQFPSSLSASVGDRVTITCRASQGIRNDLGWYQQKPGKAP | |
| | KRLIYAASRLHRGVPSRFSGSGSGTEFTLTISSLQPEDFATYYCLQ | |
| | HNSYPCSFGQGTKLEIK | |
| 49 | | |
| 50 | | |
| 51 | GGSISSSNW | |
| 52 | IYHSGST | |
| 53 | ARWTGRTDAFDI | |
| 54 | QSLLHSNGYNY | |
| 55 | LGS | |
| 56 | MQGTHWPLT | |
| 57 | QVQLQESGPGLVKPSGTLSLTCAVSGGSISSSNWWSWVRQPPGK | |
| | GLEWIGEIYHSGSTNYNPSLKSRVTISVDKSKNQFSLKLSSVTAA | |
| | DTAVYYCARWTGRTDAFDIWGQGTMVTVSS | |
| 58 | DVVMTQSPLSLPVTPGEPASISCRSSQSLLHSNGYNYLDWYLQK | |
| | PGQSPQLLIYLGSNRASGVPDRFSGSGSGTDFTLKISRVEAEDVG | |
| | VYYCMQGTHWPLTFGQGTKVEIK | |
| 59 | | |
| 60 | | |
| 61 | GFTFSSFA | |
| 62 | IDTRGAT | |
| 63 | ARLGNFYYGMDV | |
| 64 | QSIGSS | |
| 65 | YAS | |
| 66 | HQSSRLPHT | |
| 67 | EVQLVQSGGGLVKPGGSLRLSCAASGFTFSSFAMHWVRQAPGK | |
| | GLEWISVIDTRGATYYADSVKGRFTISRDNAKNSLYLQMNSLRA | |
| | EDTAVYYCARLGNFYYGMDVWGQGTTVTVSS | |
| 68 | EIVLTQSPGTLSVSPGERATLSCRASQSIGSSLHWYQQKPGQAPR | |
| | LLIKYASQSLSGIPDRFSGSGSGTDFTLTISRLEPEDFAVYYCHQS | |
| | SRLPHTFGQGTKVEIK | |
| 69 | | |
| 70 | | |
| 71 | GFTFSIYR | |
| 72 | ISPSGGTT | |
| 73 | ARWSGGSGYAFDI | |
| 74 | RDIRNY | |
| 75 | DAS | |
| 76 | QQFDSLPHT | |
| 77 | EVQLLESGGGLVQPGGSLRLSCAASGFTFSIYRMQWVRQAPGK | |
| | GLEWVSGISPSGGTTWYADSVKGRFTISRDNSKNTLYLQMNSLR | |
| | AEDTAVYYCARWSGGSGYAFDIWGQGTMVTVSS | |
| 78 | DIQMTQSPLSLSASVGDRVTITCQASRDIRNYLNWYQQKPGKAP | |
| | KLLIYDASSLQTGVPSRFGGSGSGTDFSFTIGSLQPEDIATYYCQQ | |
| | FDSLPHTFGQGTKLEIK | |
| 79 | | |
| 80 | | |
| 81 | GFTFSSYA | |
| 82 | ISGSGGTT | |
| 83 | AKDLGWSDSYYYYYGMDV | |
| 84 | QGIRND | |
| 85 | AAS | |
| 86 | LQHNSYPSS | |
| 87 | EVQLLESGGGLVQPGGSLRLSCTASGFTFSSYAMNWVRQAPGK | |
| | GLEWVSAISGSGGTTFYADSVKGRFTISRDNSRTTLYLQMNSLR | |
| | AEDTAVYYCAKDLGWSDSYYYYYGMDVWGQGTTVTVSS | |
| 88 | DIQMTQFPSSLSASVGDRVTITCRASQGIRNDLGWYQQKPGKAP | |
| | KRLIYAASRLHRGVPSRFSGSGSGTEFTLTISSLQPEDFATYYCLQ | |
| | HNSYPSSFGQGTKLEIK | |

What is claimed is:

1. A method of treating an individual with inactive or chronic thyroid eye disease (TED), the method comprising administering to an individual with inactive or chronic TED an insulin-like growth factor 1 receptor (IGF1R) inhibitor, thereby treating the inactive or chronic TED, wherein the IGF1R inhibitor is not teprotumumab.

2. The method of claim 1, wherein the inactive or chronic TED is associated with Grave's disease.

3. The method of claim 1 or 2, wherein the inactive or chronic TED has a Clinical Activity Score (CAS) of 1 or less.

4. The method of any one of claims 1 to 3, wherein the inactive or chronic TED has a Clinical Activity Score (CAS) of 0.

5. The method of any one of claims 1 to 4, wherein the inactive TED is chronic TED.

6. The method of claim 5, wherein the chronic TED has been inactive for at least 6 months prior to treatment.

7. The method of any one of claims 1 to 4, wherein the inactive or chronic TED has been inactive for at least 12 months prior to treatment.

8. The method of any one of claims 1 to 4, wherein the inactive or chronic TED has been inactive for at least 24 months prior to treatment.

9. The method of any one of claims 1 to 8, wherein the inactive or chronic TED has been previously treated with an IGF1R inhibitor.

10. The method of claim 9, wherein the IGF1R inhibitor comprises ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, A-928605, or any combination thereof.

11. The method of claim 9, wherein the IGF1R inhibitor comprises an antibody that binds IGF1R.

12. The method of any one of claims 1 to 11, wherein the IGF1R inhibitor is chimeric or humanized.

13. The method of any one of claims 1 to 12, wherein the IGF1R inhibitor is an IgG antibody.

14. The method of any one of claims 1 to 13, wherein the IGF1R inhibitor is a Fab, F(ab)₂, a single-domain antibody, or a single chain variable fragment (scFv).

15. The method of any one of claims 1 to 14, wherein the IGF1R inhibitor is an antibody comprising ganitumab, figitumumab, MEDI-573, cixutumumab, dalotuzumab, robatumumab, robatumumab, AVE1642, BIIB022, xentuzumab, istiratumab, or any combination thereof.

16. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in any one of SEQ ID NO: 11, 21, 31, 41, 51, 61, 71, or 81;
b. an immunoglobulin heavy chain CDR2 (CDR-H2) comprising the amino acid sequence set forth in any one of SEQ ID NO: 12, 22, 32, 42, 52, 62, 72, or 82;
c. an immunoglobulin heavy chain CDR3 (CDR-H3) comprising the amino acid sequence set forth in any one of SEQ ID NO: 13, 23, 33, 43, 53, 63, 73, or 83;
d. an immunoglobulin light chain CDR1 (CDR-L1) comprising the amino acid sequence set forth in any one of SEQ ID NO: 14, 24, 34, 44, 54, 64, 74, or 84;
e. an immunoglobulin light chain CDR2 (CDR-L2) amino comprising the amino acid sequence set forth in any one of SEQ ID NO: 15, 25, 35, 45, 55, 65, 75, or 85; and/or
f. and an immunoglobulin light chain CDR3 (CDR-L3) comprising the amino acid sequence set forth in any one of SEQ ID NO: 16, 26, 36, 46, 56, 66, 76, or 86.

17. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 11;
b. an immunoglobulin heavy chain CDR2 (CDR-H2) comprising the amino acid sequence set forth in SEQ ID NO: 12;
c. an immunoglobulin heavy chain CDR3 (CDR-H3) comprising the amino acid sequence set forth in SEQ ID NO: 13;
d. an immunoglobulin light chain CDR1 (CDR-L1) comprising the amino acid sequence set forth in SEQ ID NO: 14;
e. an immunoglobulin light chain CDR2 (CDR-L2) amino comprising the amino acid sequence set forth in SEQ ID NO: 15; and/or
f. and an immunoglobulin light chain CDR3 (CDR-L3) comprising the amino acid sequence set forth in SEQ ID NO: 16.

18. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 21;
b. an immunoglobulin heavy chain CDR2 (CDR-H2) comprising the amino acid sequence set forth in SEQ ID NO: 22;
c. an immunoglobulin heavy chain CDR3 (CDR-H3) comprising the amino acid sequence set forth in SEQ ID NO: 23;
d. an immunoglobulin light chain CDR1 (CDR-L1) comprising the amino acid sequence set forth in SEQ ID NO: 24;
e. an immunoglobulin light chain CDR2 (CDR-L2) amino comprising the amino acid sequence set forth in SEQ ID NO: 25; and/or
f. and an immunoglobulin light chain CDR3 (CDR-L3) comprising the amino acid sequence set forth in SEQ ID NO: 26.

19. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 31;
b. an immunoglobulin heavy chain CDR2 (CDR-H2) comprising the amino acid sequence set forth in SEQ ID NO: 32;
c. an immunoglobulin heavy chain CDR3 (CDR-H3) comprising the amino acid sequence set forth in SEQ ID NO: 33;
d. an immunoglobulin light chain CDR1 (CDR-L1) comprising the amino acid sequence set forth in SEQ ID NO: 34;
e. an immunoglobulin light chain CDR2 (CDR-L2) amino comprising the amino acid sequence set forth in SEQ ID NO: 35; and/or
f. and an immunoglobulin light chain CDR3 (CDR-L3) comprising the amino acid sequence set forth in SEQ ID NO: 36.

20. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 41;
b. an immunoglobulin heavy chain CDR2 (CDR-H2) comprising the amino acid sequence set forth in SEQ ID NO: 42;
c. an immunoglobulin heavy chain CDR3 (CDR-H3) comprising the amino acid sequence set forth in SEQ ID NO: 43;
d. an immunoglobulin light chain CDR1 (CDR-L1) comprising the amino acid sequence set forth in SEQ ID NO: 44;
e. an immunoglobulin light chain CDR2 (CDR-L2) amino comprising the amino acid sequence set forth in SEQ ID NO: 45; and/or
f. and an immunoglobulin light chain CDR3 (CDR-L3) comprising the amino acid sequence set forth in SEQ ID NO: 46.

21. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 51;
b. an immunoglobulin heavy chain CDR2 (CDR-H2) comprising the amino acid sequence set forth in SEQ ID NO: 52;
c. an immunoglobulin heavy chain CDR3 (CDR-H3) comprising the amino acid sequence set forth in SEQ ID NO: 53;
d. an immunoglobulin light chain CDR1 (CDR-L1) comprising the amino acid sequence set forth in SEQ ID NO: 54;
e. an immunoglobulin light chain CDR2 (CDR-L2) amino comprising the amino acid sequence set forth in SEQ ID NO: 55; and/or
f. and an immunoglobulin light chain CDR3 (CDR-L3) comprising the amino acid sequence set forth in SEQ ID NO: 56.

22. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 61;
b. an immunoglobulin heavy chain CDR2 (CDR-H2) comprising the amino acid sequence set forth in SEQ ID NO: 62;
c. an immunoglobulin heavy chain CDR3 (CDR-H3) comprising the amino acid sequence set forth in SEQ ID NO: 63;
d. an immunoglobulin light chain CDR1 (CDR-L1) comprising the amino acid sequence set forth in SEQ ID NO: 64;
e. an immunoglobulin light chain CDR2 (CDR-L2) amino comprising the amino acid sequence set forth in SEQ ID NO: 65; and/or
f. and an immunoglobulin light chain CDR3 (CDR-L3) comprising the amino acid sequence set forth in SEQ ID NO: 66.

23. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 71;
b. an immunoglobulin heavy chain CDR2 (CDR-H2) comprising the amino acid sequence set forth in SEQ ID NO: 72;
c. an immunoglobulin heavy chain CDR3 (CDR-H3) comprising the amino acid sequence set forth in SEQ ID NO: 73;
d. an immunoglobulin light chain CDR1 (CDR-L1) comprising the amino acid sequence set forth in SEQ ID NO: 74;
e. an immunoglobulin light chain CDR2 (CDR-L2) amino comprising the amino acid sequence set forth in SEQ ID NO: 75; and/or
f. and an immunoglobulin light chain CDR3 (CDR-L3) comprising the amino acid sequence set forth in SEQ ID NO: 76.

24. The method of any one of claims 1 to 14, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 81;
b. an immunoglobulin heavy chain CDR2 (CDR-H2) comprising the amino acid sequence set forth in SEQ ID NO: 82;
c. an immunoglobulin heavy chain CDR3 (CDR-H3) comprising the amino acid sequence set forth in SEQ ID NO: 83;
d. an immunoglobulin light chain CDR1 (CDR-L1) comprising the amino acid sequence set forth in SEQ ID NO: 84;
e. an immunoglobulin light chain CDR2 (CDR-L2) amino comprising the amino acid sequence set forth in SEQ ID NO: 85; and/or
f. and an immunoglobulin light chain CDR3 (CDR-L3) comprising the amino acid sequence set forth in SEQ ID NO: 86.

25. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18.

26. The method of any one of claims 1 to 14, wherein the IGF1R inhibitor is an antibody or and an immunoglobulin light chain variable region, wherein the immunoglobulin heavy chain variable region comprises an amino acid sequence at least about 90%, 95%, 97%, 99%, or 100% identical to that set forth in SEQ ID NO: 27; and wherein the immunoglobulin light chain variable region comprises an amino acid sequence at least about 90%, 95%, 97%, 99%, or 100% identical to that set forth in SEQ ID NO: 28.

27. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 37; and wherein the immunoglobulin light chain variable region comprises an amino acid sequence at least about 90%, 95%, 97%, 99%, or 100% identical to that set forth in SEQ ID NO: 38.

28. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 47; and wherein the immunoglobulin light chain variable region comprises an amino acid sequence at least about 90%, 95%, 97%, 99%, or 100% identical to that set forth in SEQ ID NO: 48.

29. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 57; and wherein the immunoglobulin light chain variable region comprises an amino acid sequence at least about 90%, 95%, 97%, 99%, or 100% identical to that set forth in SEQ ID NO: 58.

30. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 67; and wherein the immunoglobulin light chain variable region comprises an amino acid sequence at least about 90%, 95%, 97%, 99%, or 100% identical to that set forth in SEQ ID NO: 68.

31. The method of any one of claims 1 to 14, wherein the IGF1R inhibitor is an antibody or and an immunoglobulin light chain variable region, wherein the immunoglobulin heavy chain variable region comprises an amino acid sequence at least about 90%, 95%, 97%, 99%, or 100% identical to that set forth in SEQ ID NO: 77; and wherein the immunoglobulin light chain variable region comprises an amino acid sequence at least about 90%, 95%, 97%, 99%, or 100% identical to that set forth in SEQ ID NO: 78.

32. The method of any one of claims 1 to 14, wherein the IGF1R inhibitor is an antibody or antigen binding fragment thereof comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 87; and wherein the immunoglobulin light chain variable region comprises an amino acid sequence at least about 90%, 95%, 97%, 99%, or 100% identical to that set forth in SEQ ID NO: 88.

33. The method of any one of claims 1 to 14, wherein the IGF1R inhibitor is an antibody or antigen binding fragment thereof comprising an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 9; and wherein the immunoglobulin light chain comprises an amino acid sequence at least about 90%, 95%, 97%, 99%, or 100% identical to that set forth in SEQ ID NO: 10.

34. The method of any one of claims 1 to 33, wherein the IGF1R inhibitor inhibits IGF1R signaling.

35. The method of any one of claims 1 to 34, wherein the IGF1R inhibitor is administered at a dose of about 5 mg/kg to about 50 mg/kg.

36. The method of any one of claims 1 to 35, wherein the IGF1R inhibitor is administered at a first dose of about 10 mg/kg and at a subsequent dose of about 20 mg/kg.

37. The method of any one of claims 1 to 36, wherein the IGF1R inhibitor is administered once every three weeks.

38. The method of any one of claims 1 to 37, wherein the IGF1R inhibitor is included in a pharmaceutical formulation comprising a pharmaceutically acceptable excipient, carrier, or diluent.

39. The method of claim 38, wherein the pharmaceutical formulation is formulated for intravenous administration.

40. The method of claim 38, wherein the pharmaceutical formulation is formulated for subcutaneous administration.

41. The method of any one of claims 1 to 40, wherein the IGF1R inhibitor reduces proptosis in the individual with inactive or chronic TED.

42. The method of claim 41, wherein the IGF1R inhibitor reduces proptosis by at least about 2 mm in the individual with inactive or chronic TED.

43. The method of claim 41, wherein the IGF1R inhibitor reduces proptosis by at least about 3 mm in the individual with inactive or chronic TED.

44. The method of any one of claims 1 to 43, wherein the IGF1R inhibitor reduces diplopia in the individual with inactive or chronic TED.

45. The method of claim 44, wherein the IGF1R inhibitor reduces a diplopia score in the individual with inactive or chronic TED by at least 2 grades.

46. The method of claim 44, wherein the IGF1R inhibitor reduces a diplopia score in the individual with inactive or chronic TED to 0.

47. The method of any one of claims 1 to 46, wherein the IGF1R inhibitor reduces binocular diplopia in the individual with inactive or chronic TED.

48. The method of claim 47, wherein the IGF1R inhibitor reduces a binocular diplopia score in the individual with inactive or chronic TED by at least 2 grades.

49. The method of claim 47, wherein the IGF1R inhibitor reduces a binocular diplopia score in the individual with inactive or chronic TED to 0.

50. The method of any one of claims 1 to 49 wherein the individual with the inactive or chronic TED has not been previously treated with an IGF1R inhibitor.

51. The method of any one of claims 1 to 50, wherein the individual with the inactive or chronic TED has not previously undergone orbital irradiation.

52. The method of any one of claims 1 to 51, wherein the individual with the inactive or chronic TED has not previously undergone orbital decompression surgery.

53. The method of any one of claims 1 to 52, wherein the individual with the inactive or chronic TED has not previously undergone strabismus surgery.

54. A method of improving a quality of life of an individual with inactive thyroid eye disease (TED), comprising administering to the individual with inactive TED an insulin-like growth factor 1 receptor (IGF1R) inhibitor, thereby improving the quality of the life of the individual with inactive TED, wherein the quality of life is measured by an increased score on the Graves' Ophthalmopathy Quality of Life (GO-QoL) questionnaire.

55. The method of claim 54, wherein a score of the individual with inactive TED on the GO-QoL questionnaire is increased by at least 5 points.

56. The method of claim 54, wherein a score of the individual with inactive TED on the GO-QoL questionnaire is increased by at least 8 points.

57. The method of claim 54, wherein a score of the individual with inactive TED on the GO-QoL questionnaire is increased by at least 10 points.

58. The method of claim 54, wherein a score of the individual with inactive TED on the GO-QoL questionnaire is increased by at least 15 points.

59. The method of claim 54, wherein a score of the individual with inactive TED on the GO-QoL questionnaire is increased by at least 20 points.

60. The method of any one of claims 54-59, wherein the GO-QoL questionnaire comprises a visual functioning subscale.

61. The method of any one of claims 54-60, wherein the GO-QoL questionnaire comprises an appearance subscale.

62. The method of any one of claims 54-61, wherein the individual with inactive TED has a clinical activity score (CAS) of 2 or less.

63. The method of any one of claims 54-62, wherein the individual with inactive TED has a clinical activity score (CAS) of 1 or less.

64. The method of any one of claims 54-63, wherein the individual with inactive TED has a clinical activity score (CAS) of 0.

65. The method of any one of claims 54-64, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 1;
b. an immunoglobulin heavy chain CDR2 (CDR-H2) comprising the amino acid sequence set forth in SEQ ID NO: 2;
c. an immunoglobulin heavy chain CDR3 (CDR-H3) comprising the amino acid sequence set forth in SEQ ID NO: 3;
d. an immunoglobulin light chain CDR1 (CDR-L1) comprising the amino acid sequence set forth in SEQ ID NO: 4;
e. an immunoglobulin light chain CDR2 (CDR-L2) amino comprising the amino acid sequence set forth in SEQ ID NO: 5; and/or
f. and an immunoglobulin light chain CDR3 (CDR-L3) comprising the amino acid sequence set forth in SEQ ID NO: 6.

66. The method of any one of claims 54-65, wherein the IGF1R inhibitor is teprotumumab.

67. The method of any one of claims 54-66, wherein the IGF1R inhibitor is administered at a dose of about 5 mg/kg to about 50 mg/kg.

68. The method of any one of claims 54-67, wherein the IGF1R inhibitor is administered at a first dose of about 10 mg/kg and at a subsequent dose of about 20 mg/kg.

69. The method of any one of claims 54-68, wherein the IGFR inhibitor reduces proptosis by at least about 1 mm in the individual with inactive TED.

70. The method of any one of claims 54-69, wherein the IGFR inhibitor reduces proptosis by at least about 2 mm in the individual with inactive TED.

71. The method of any one of claims 54-70, wherein the IGFR inhibitor reduces a diplopia score in the individual with inactive TED by at least 2 grades.

72. The method of any one of claims 54-70, wherein the IGFR inhibitor reduces a diplopia score in the individual with inactive TED by at least 1 grade.

73. The method of any one of claims 54-70, wherein the IGFR inhibitor reduces a diplopia score in the individual with inactive TED to 0.

74. The method of any one of claims 54-73, wherein the individual with inactive TED has not been previously treated with an IGF1R inhibitor.

75. The method of any one of claims 54-74, wherein the individual with inactive TED has not previously undergone orbital irradiation.

76. The method of any one of claims 54-75, wherein the individual with inactive TED has not previously undergone orbital decompression surgery.

77. The method of any one of claims 54-76, wherein the individual with inactive TED has not previously undergone strabismus surgery.

78. A method of improving a quality of life of an individual with chronic thyroid eye disease (TED), comprising administering to the individual with chronic TED an insulin-like growth factor 1 receptor (IGF1R) inhibitor, thereby improving the quality of the life of the individual with chronic TED, wherein the quality of life is measured by an increased score on the Graves' Ophthalmopathy Quality of Life (GO-QoL) questionnaire.

79. The method of claim 78, wherein a score of the individual with chronic TED on the GO-QoL questionnaire is increased by at least 5 points.

80. The method of claim 78, wherein a score of the individual with chronic TED on the GO-QoL questionnaire is increased by at least 8 points.

81. The method of claim 78, wherein a score of the individual with chronic TED on the GO-QoL questionnaire is increased by at least 10 points.

82. The method of claim 78, wherein a score of the individual with chronic TED on the GO-QoL questionnaire is increased by at least 15 points.

83. The method of claim 78, wherein a score of the individual with chronic TED on the GO-QoL questionnaire is increased by at least 20 points.

84. The method of claim any one of claims 78-83, wherein the GO-QoL questionnaire comprises a visual functioning subscale.

85. The method of claim any one of claims 78-84, wherein the GO-QoL questionnaire comprises an appearance subscale.

86. The method of any one of claims 78-85, wherein the individual with chronic TED has had the chronic TED for 6 months or longer.

87. The method of any one of claims 78-86, wherein the individual with chronic TED has had the chronic TED for 12 months or longer.

88. The method of any one of claims 78-87, wherein the individual with chronic TED has had the chronic TED for 18 months or longer.

89. The method of any one of claims 78-88, wherein the individual with chronic TED has had the chronic TED for 24 months or longer.

90. The method of any one of claims 78-89, wherein the individual with chronic TED has had the chronic TED for 36 months or longer.

91. The method of any one of claims 78-90, wherein the individual with chronic TED has had the chronic TED for 48 months or longer.

92. The method of any one of claims 78-91, wherein the individual with chronic TED has had the chronic TED for 100 months or longer.

93. The method of any one of claims 78-92, wherein the IGF1R inhibitor is an antibody or antigen binding fragment thereof comprising:
a. an immunoglobulin heavy chain CDR1 (CDR-H1) comprising the amino acid sequence set forth in SEQ ID NO: 1;
b. an immunoglobulin heavy chain CDR2 (CDR-H2) comprising the amino acid sequence set forth in SEQ ID NO: 2;
c. an immunoglobulin heavy chain CDR3 (CDR-H3) comprising the amino acid sequence set forth in SEQ ID NO: 3;
d. an immunoglobulin light chain CDR1 (CDR-L1) comprising the amino acid sequence set forth in SEQ ID NO: 4;
e. an immunoglobulin light chain CDR2 (CDR-L2) amino comprising the amino acid sequence set forth in SEQ ID NO: 5; and/or
f. and an immunoglobulin light chain CDR3 (CDR-L3) comprising the amino acid sequence set forth in SEQ ID NO: 6.

94. The method of any one of claims 78-93, wherein the IGF1R inhibitor is teprotumumab.

95. The method of any one of claims 78-94, wherein the IGF1R inhibitor is administered at a dose of about 5 mg/kg to about 50 mg/kg.

96. The method of any one of claims 78-95, wherein the IGF1R inhibitor is administered at a first dose of about 10 mg/kg and at a subsequent dose of about 20 mg/kg.

97. The method of any one of claims 78-96, wherein the IGFR inhibitor reduces proptosis by at least about 1 mm in the individual with chronic TED.

98. The method of any one of claims 78-97, wherein the IGFR inhibitor reduces proptosis by at least about 2 mm in the individual with chronic TED.

99. The method of any one of claims 78-98, wherein the IGFR inhibitor reduces a diplopia score in the individual with chronic TED by at least 2 grades.

100. The method of any one of claims 78-99, wherein the IGFR inhibitor reduces a diplopia score in the individual with chronic TED by at least 1 grade.

101. The method of any one of claims 78-100, wherein the IGFR inhibitor reduces a diplopia score in the individual with chronic TED to 0.

102. The method of any one of claims 78-101, wherein the individual with chronic TED has not been previously treated with an IGF1R inhibitor.

103. The method of any one of claims 78-102, wherein the individual with chronic TED has not previously undergone orbital irradiation.

104. The method of any one of claims 78-103, wherein the individual with chronic TED has not previously undergone orbital decompression surgery.

105. The method of any one of claims 78-104, wherein the individual with chronic TED has not previously undergone strabismus surgery.
